SEQUENCE LISTING (20 sequences)

SEQ ID NO: 1 - protein, 9 aa, Homo sapiens
EEQYNSTYR

SEQ ID NO: 2 - protein, 9 aa, Homo sapiens
EEQFNSTFR

SEQ ID NO: 3 - protein, 9 aa, Homo sapiens
EEQYNSTFR

SEQ ID NO: 4 - protein, 9 aa, Homo sapiens
EEQFNSTYR

SEQ ID NO: 5 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 385 aa, Homo sapiens
MPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 7 - protein, 357 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFRLVFRGMSISRPNAVVGRCRMIRHSRDKKNEPNPQRFDRIAHTKETMLSDGLNSLTYQVLDVQRYPLYTQITVDIGTPS

SEQ ID NO: 8 - protein, 225 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQYEKIRRLLW

SEQ ID NO: 9 - protein, 24 aa, Homo sapiens
MRLREPLLSGSAAMPGASLQRACR

SEQ ID NO: 10 - protein, 20 aa, Homo sapiens
LLVAVCALHLGVTLVYYLAG

SEQ ID NO: 11 - protein, 354 aa, Homo sapiens
RDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 12 - protein, 290 aa, Artificial
  B4GalT
GPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 13 - protein, 308 aa, Artificial
  B4GalT
GSSPLLDMGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPRDHHHHHHH

SEQ ID NO: 14 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 15 - protein, 175 aa, Homo sapiens
MNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 16 - protein, 9 aa, Homo sapiens
MIHTNLKKK

SEQ ID NO: 17 - protein, 17 aa, Homo sapiens
FSCCVLVFLLFAVICVW

SEQ ID NO: 18 - protein, 380 aa, Homo sapiens
KEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 19 - protein, 320 aa, Artificial
  ST6Gal1
AKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 20 - protein, 339 aa, Artificial
  ST6Gal1
GSSPLLDMLEHHHHHHHHMAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

(19) United States
(12) Patent Application Publication
(10) Pub. No.: US 2023/0192814 A1
(43) Pub. Date: Jun. 22, 2023

(54) HYPER-SIALYLATED IMMUNOGLOBULIN

(71) Applicant: Momenta Pharmaceuticals, Inc., Titusville, NJ (US)

(72) Inventor: John Schaeck, Somerville, MA (US)

(21) Appl. No.: 17/925,999

(22) PCT Filed: May 19, 2021
(86) PCT No.: PCT/US2021/033150

§ 371 Date: Nov. 17, 2022

Related U.S. Application Data
(60) Provisional application No. 63/108,741, filed on Nov. 2, 2020.
(60) Provisional application No. 63/026,826, filed on May 19, 2020.

(51) Int. Cl. C07K 16/06; C12N 9/10

(52) U.S. Cl. CPC C07K 16/06; C12N 9/1051; C12Y 204/01038; C12N 9/1048; C07K 2317/41; C07K 2317/55; C07K 2317/52

(57) ABSTRACT

Methods for preparing hypersialylated IgG are described.

### CLAIM OF PRIORITY

This application claims the benefit of U.S. Provisional Application Ser. No. 63/026,826, filed on May 19, 2020, and 63/108,741, filed Nov. 2, 2020. The entire contents of the foregoing are incorporated herein by reference.

### TECHNICAL FIELD

The present disclosure relates to methods for preparing hypersialylated IgG.

### BACKGROUND

Intravenous immunoglobulin (IVIg), which is prepared from the pooled plasma of human donors (e.g., pooled plasma from at least 1,000 donors), is used to treat a variety of inflammatory disorders. However, IVIg preparations have distinct limitations, such as variable efficacy, clinical risks, high costs, and finite supply. Different IVIg preparations are frequently treated as interchangeable products clinically, but it is well-known that significant differences in product preparations exist that may impact tolerability and activity in selected clinical applications. At the current maximal dosing regimens, only partial and unsustained responses are obtained in many instances. In addition, the long infusion times (4-6 h) associated with the high volume of IVIg treatment consume significant resources at infusion centers and negatively affect patient-reported outcomes, such as convenience and quality of life.

The identification of the important anti-inflammatory role of Fc domain sialylation has presented an opportunity to develop more potent immunoglobulin therapies. Commercially available IVIg preparations generally exhibit low levels of sialylation on the Fc domain of the antibodies present. Specifically, they exhibit low levels of di-sialylation of the branched glycans on the Fc region.

Washburn et al. (Proceedings of the National Academy of Sciences, USA 112: E1297-E1306 (2015)) describes a controlled sialylation process to generate highly tetra-Fc-sialylated IVIg and showed that the process yields a product with consistent enhanced anti-inflammatory activity.

### SUMMARY

The sialylation reaction driven by ST6Gal using CMP-NANA as a substrate has characteristics that make improvement of the reaction, whether assessed by overall level of disialylation, time to reach a certain level of disialylation, amount of enzyme and substrate required to reach a certain overall level of disialylation, challenging. For example: (a) CMP-NANA is not entirely stable and will spontaneously hydrolyze even in the absence of any enzyme; (b) ST6Gal1 is thought to catalyze hydrolysis of the CMP-NANA without productive addition to the Gal on a branched glycan; (c) cytidine monophosphate (CMP), a side-product generated either through enzymatic addition or CMP-NANA hydrolysis, can act as a competitive inhibitor of ST6Gal1; (d) CMP has been observed to catalyze the reverse enzymatic reaction to remove the NeuAc from the newly formed glycan. Thus, over time the level of side-products will increase and this can lead to slowing or even reversal of the desired sialylation reaction.

The present disclosure is based, at least in part, on the discovery that the reverse reaction is far less favorable with BIS-TRIS as buffer, e.g., as when compared to MOPS as buffer.

Thus, described herein are methods of producing hypersialylated IgG (hsIgG) comprising: (a) providing pooled IgG antibodies; (b) incubating the pooled IgG antibodies in a reaction mixture comprising β1,4-Galactosyltransferase (B4GalT) or enzymatically active portion thereof, UDP-Gal or salt thereof, Bis (2-hydroxyethyl) aminotris (hydroxymethyl)methane (BIS-TRIS) buffer, and MnCl₂, thereby producing galactosylated IgG antibodies; and (c) incubating the galactosylated IgG antibodies in a reaction mixture comprising ST6Gal or enzymatically active portion thereof, CMP-NANA or salt thereof, Bis (2-hydroxyethyl) aminotris (hydroxymethyl)methane (BIS-TRIS) buffer, and MnCl₂, thereby producing hsIgG.

Also described herein are methods of preparing hypersialylated IgG (hsIgG) comprising: (a) providing pooled IgG antibodies; (b) incubating the pooled IgG antibodies in a reaction mixture comprising β1,4-Galactosyltransferase (B4GalT) or enzymatically active portion thereof, UDP-Gal or salt thereof, ST6Gal or enzymatically active portion thereof, CMP-NANA or salt thereof, Bis (2-hydroxyethyl) aminotris (hydroxymethyl)methane (BIS-TRIS) buffer, and MnCl₂, thereby creating the hsIgG preparation.

Also described herein are methods of preparing hypersialylated IgG (hsIgG) comprising: (a) providing pooled IgG antibodies; (b) incubating the pooled IgG antibodies in a galactosylation reaction mixture comprising β1,4-Galactosyltransferase (B4GalT) or enzymatically active portion thereof, UDP-Gal or salt thereof, Bis (2-hydroxyethyl) aminotris (hydroxymethyl)methane (BIS-TRIS) buffer, and MnCl₂, thereby producing galactosylated IgG antibodies; (c) adding ST6Gal or an enzymatically active portion thereof and CMP-NANA or salt thereof to the galactosylation reaction mixture to produce a sialylation reaction mixture; and (d) incubating the sialylation reaction mixture, thereby producing hsIgG

In some embodiments, the B4GalT or enzymatically active portion thereof is at least 85% identical to SEQ ID NO: 13.

In some embodiments, the ST6Gal or enzymatically active portion thereof comprises an amino acid sequence that is at least 90% identical to SEQ ID NO: 19.

In some embodiments, the total incubation time is less than 72 hours.

In some embodiments, the incubation time of the reaction mixture comprising ST6Gal or enzymatically active portion thereof is less than 40 hours.

In some embodiments, each of the reaction mixture(s) each independently comprise BIS-TRIS at from about 10 to about 500 mM and from about pH 5.5 to about pH 8.5.

In some embodiments, the reaction mixture(s) each independently comprise BIS-TRIS buffer at about 50 mM and about pH 7.3.

In some embodiments, the pooled IgG antibodies are provided as a composition further comprising BIS-TRIS buffer at about pH 7.2.

In some embodiments, each of the reaction mixture(s) each independently comprise MnCl₂ at about 1 to about 20 mM.

In some embodiments, each of the reaction mixture(s) each independently comprise MnCl₂ at about 4.5 to about 5.5 mM.

In some embodiments, the reaction mixture comprises from about 0.038 to about 0.046 UDP-Gal or salt thereof per gram of pooled IgG antibodies.

In some embodiments, the reaction mixture comprises about 0.1425 to about 0.1575 CMP-NANA or salt thereof per gram of IgG antibody.

In some embodiments, the reaction mixture comprising CMP-NANA is supplemented with additional CMP-NANA or salt thereof during incubation.

In some embodiments, the total amount of CMP-NANA or salt thereof added to the reaction mixture comprising CMP-NANA is from about 0.1425 to about 0.1575.

In some embodiments, the total amount of CMP-NANA is added to the sialylation reaction mixture in less than 7 portions.

In some embodiments, the reaction mixture comprising B4GalT or enzymatically active portion thereof comprises from about 7.2 to or to about 8.8 U B4GalT or enzymatically active portion thereof per gram of pooled IgG.

In some embodiments, the reaction mixture comprising ST6Gal1 or enzymatically active portion thereof comprises from about 17.1 to about 18.9 U ST6Gal1 or enzymatically active portion thereof per gram of pooled IgG.

In some embodiments, the incubation takes place at from about 20 to about 50° C.

In some embodiments, the incubation takes place at about 37° C.

In some embodiments, the IgG antibodies comprise IgG antibodies isolated from at least 1000 donors.

In some embodiments, at least 50%, 55%, 60%, 65% or 70% w/w of the IgG antibodies are IgG1 antibodies.

In some embodiments, at least 90% of the donor subject has been exposed to a virus.

In some embodiments, about 60%, 65%, 70%, 75%, 80%, or 85% of the branched glycans on the hsIgG have a sialic acid on both the α1,3 branch and the α1,6 branch.

In some embodiments, about 60%, 65%, 70%, 75%, 80%, or 85% of the branched Fc glycans on the hsIgG have a sialic acid on both the α1,3 branch and the α1,6 branch.

In some embodiments, at least 60%, 65%, 70%, 75%, 80%, or 85% of the branched glycans on the Fab domain of the hsIgG have a sialic acid on both the α1,3 arm and the α 1,6 arm that is connected through a NeuAc-α 2,6-Gal terminal linkage.

In some embodiments, at least 80% of the branched Fc glycans on the hsIgG have a sialic acid on both the α1,3 branch and the α1,6 branch.

In some embodiments, at least 60%, 65%, 70% of the branched glycans on the Fab domain of the hsIgG have a sialic acid on both the α1,3 arm and the α1,6 arm that is connected through a NeuAc-α 2,6-Gal terminal linkage.

In some embodiments, at least 85% of the branched Fc glycans on the hsIgG have a sialic acid on both the α1,3 branch and the α1,6 branch.

In some embodiments, at least 60%, 65%, 70% of the branched glycans on the Fab domain of the hsIgG have a sialic acid on both the α1,3 arm and the α1,6 arm that is connected through a NeuAc-α 2,6-Gal terminal linkage.

In some embodiments, at least 90% of the branched Fc glycans on the hsIgG have a sialic acid on both the α1,3 branch and the α1,6 branch.

In some embodiments, at least 60%, 65%, 70% of the branched glycans on the Fab domain of the hsIgG have a sialic acid on both the α1,3 arm and the α1,6 arm that is connected through a NeuAc-α 2,6-Gal terminal linkage.

Also described herein are methods for preparing immunoglobulin G (IgG) having a very high level of Fc sialylation, particularly disialylation (sialylation on both the alpha 1,3 branch and the alpha 1,6 branch of the glycan at Asn297 (EU Numbering). The methods described herein can provide hypersialylated IgG (hsIgG) in which greater than 70% of the branched glycans on the Fc domain are sialylated on both branches (i.e., on the alpha 1,3 branch and the alpha 1,6 branch). HsIgG contains a diverse mixture of IgG antibodies, primarily IgG1 antibodies. The diversity of the antibodies is high. The immunoglobulins used to prepare hsIgG can be obtained, for example from pooled human plasma (e.g., pooled plasma from at least 1,000-30,000 donors). The immunoglobulins can be obtained from IVIg, including commercially available IVIg. HsIgG has far higher level of sialic acid on the branched glycans on the Fc region than does IVIg. This results in a composition that differs from IVIg in both structure and activity. HsIgG can be prepared as described in WO2014/179601 or Washburn et al. (Proceedings of the National Academy of Sciences, USA 112: E1297-E1306 (2015)), both of which are hereby incorporated by reference.

Described herein are improved methods for preparing hsIgG.

Described herein is a method of preparing hypersialylated (hsIgG), the method comprising: (a) providing a mixture of IgG antibodies; (b) incubating the mixture of IgG antibodies in a reaction mixture comprising β1,4-Galactosyltransferase I (B4GalT) and UDP-Gal to produce galactosylated IgG antibodies; (c) incubating the galactosylated IgG antibodies in a reaction mixture comprising ST6Gal1 and CMP-NANA, wherein the galactosylation reaction mixture and the sialylation reaction mixture comprise Bis (2-hydroxyethyl) aminotris (hydroxymethyl)methane (BIS-TRIS) buffer, thereby creating the hsIgG preparation.

Also described is a method of preparing hypersialylated (hsIgG), the method comprising (a) providing a mixture of IgG antibodies; (b) incubating the mixture of IgG antibodies in a reaction mixture comprising β1,4-Galactosyltransferase I (B4GalT), UDP-Gal, ST6Gal1 and CMP-NANA, in Bis (2-hydroxyethyl) aminotris (hydroxymethyl)methane (BIS-TRIS) buffer, for at least 24 hours, thereby creating the hsIgG preparation.

In various embodiments: the B4GalT is at least 85% identical to SEQ ID NO: 13; the ST6Gal1 comprises an amino acid sequence that is at least 90% identical to SEQ ID NO: 19; step (b) is carried out for at least 8, 12, 18, 24, 30, or 40 hrs; step (c) is carried out for at least 8, 12, 18, 24, 30, or 40 hrs; step (c) comprises adding ST6Gal1 and CMP-NANA to the reaction mixture of step (a); the reactions take place in BIS-TRIS at 10-500 mM pH 5.5-8.5; the reaction mixtures comprise MnCl₂ at 1-20 mM; the UDP-Gal is present at 5 μM UDP-Gal/g IgG antibody; the CMP-NANA is present at 5 μM CMP-NANA/g IgG antibody; the incubation takes place at 20-50° C.; the incubation takes place at 30-45° C.; the IgG antibodies comprise IgG antibodies isolated from at least 1000 donors; at least 50%, 55%, 60%, 65% or 70% w/w of the IgG antibodies are IgG1 antibodies; at least 90% of the donor subject has been exposed to a virus; about 60%, 65%, 70%, 75%, 80%, or 85% of the branched glycans in the hsIgG preparation have a sialic acid on both the α1,3 branch and the α1,6 branch; about 60%, 65%, 70%, 75%, 80%, or 85% of the branched Fc glycans in the hsIgG preparation have a sialic acid on both the α1,3 branch and the α1,6 branch; at least 60%, 65%, 70%, 75%, 80%, or 85% of the branched glycans on the Fab domain have a sialic acid on both the α1,3 arm and the α1,6 arm that is connected through a NeuAc-α 2,6-Gal terminal linkage; at least 60%, 65%, 70%, 75%, 80%, or 85% of the branched glycans on the Fc domain have a sialic acid on both the α1,3 arm and the α1,6 arm that is connected through a NeuAc-α 2,6-Gal terminal linkage; the incubation in step (a) is 12-30 hours; and the incubation in step (a) is 20-40 hours.

In hypersialylated IgG at least 60% (e.g., 65%, 70%, 75%, 80%, 82%, 85%, 87%, 90%, 92%, 94%, 95%, 97%, 98% up to and including 100%) of branched glycans on the Fc region are di-sialylated (i.e., on both the α1,3 branch and the α1,6 arm) by way of NeuAc-α 2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (i.e., sialylated only on the α1,3 branch or only on the α1,6 branch) by way of a NeuAc-α 2,6-Gal terminal linkage.

In some embodiments, the polypeptides are derived from plasma, e.g., human plasma. In certain embodiments, the polypeptides are overwhelmingly IgG polypeptides (e.g., IgG1, IgG2, IgG3 or IgG4 or mixtures thereof), although trace amounts of other contain trace amount of other immunoglobulin subclasses can be present.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

As used herein, the term “constant region” refers to a polypeptide that corresponds to, or is derived from, one or more constant region immunoglobulin domains of an antibody. A constant region can include any or all of the following immunoglobulin domains: a C_{H}1 domain, a hinge region, a C_{H}2 domain, a C_{H}3 domain (derived from an IgA, IgD, IgG, IgE, or IgM), and a C_{H}4 domain (derived from an IgE or IgM).

As used herein, the term “Fc region” refers to a dimer of two “Fc polypeptides,” each “Fc polypeptide” including the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting P232, to its carboxyl-terminus, wherein the numbering is according to the EU system (Edelman et al., Proc. Natl. Acad. USA, 63, 78-85 (1969)). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIg.

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

As used herein, “IVIg” is a preparation of pooled, polyvalent IgG, including all four IgG subgroups, extracted from plasma of at least 1,000 human donors. IVIg is approved as a plasma protein replacement therapy for immune deficient patients. The level of IVIg Fc glycan sialylation varies among IVIg preparations, but is generally less than 20%. The level of disialylation is generally far lower than 20%. As used herein, the term “derived from IVIg” refers to polypeptides which result from manipulation of IVIg. For example, polypeptides purified from IVIg (e.g., enriched for sialylated IgGs or modified IVIg (e.g., IVIg IgGs enzymatically sialylated).

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal1 and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 branch or an α1,6 branch. The term “di-sialylated” refers to a branched glycan having a terminal sialic acid on two arms, e.g., both an α1,3 arm and an α1,6 arm.

### DESCRIPTION OF DRAWINGS

FIG. 1 shows a short, branched core oligosaccharide comprising two N-acetylglucosamine and three mannose residues. One of the branches is referred to in the art as the “α1,3 arm,” and the second branch is referred to as the “α1,6 arm,”. Squares: N-acetylglucosamine; dark gray circles: mannose; light gray circles: galactose; diamonds: N-acetylneuraminic acid; triangles: fucose.

FIG. 2 shows common Fc glycans present in IVIg. Squares: N-acetylglucosamine; dark gray circles: mannose; light gray circles: galactose; diamonds: N-acetylneuraminic acid; triangles: fucose.

FIG. 3 shows how immunoglobulins, e.g., IgG antibodies, can be sialylated by carrying out a galactosylation step followed by a sialylation step. Squares: N-acetylglucosamine; dark gray circles: mannose; light gray circles: galactose; diamonds: N-acetylneuraminic acid; triangles: fucose.

FIG. 4 shows the reaction product of a representative example of the IgG-Fc glycan profile for a reaction starting with IVIg. The left panel is a schematic representation of enzymatic sialylation reaction to transform IgG to hsIgG; the right panel is the IgG Fc glycan profile for the starting IVIg and hsIgG. Bars, from left to right, correspond to IgG1, IgG2/3, and IgG3/4, respectively.

FIG. 5 shows the level of A2F formation as a result of sialylation a Fc containing protein with various buffers at various pHs.

FIG. 6 shows the level of 1,6-A1F formation as a result of sialylation a Fc containing protein with various buffers at various pHs.

FIG. 7A shows the effect of high MnCl₂ concentration on galactosylation and sialylation of IVIg. Bars, from left to right: G1F+NeuAc; G1+NeuAc.

FIG. 7B shows the effect of high MnCl₂ concentration on galactosylation and sialylation of IVIg. Bars, from left to right: 5 mM; 10 mM; 20 mM; 40 mM; 61 mM.

FIG. 8 shows the effect of high MnCl₂ concentration on disialylation of IVIg.

FIG. 9 shows the effect of ≤10 mM MnCl₂ concentration on galactosylation of IVIg (grouped by MnCl₂ concentration).

FIG. 10 shows the effect of ≤10 mM MnCl₂ concentration on galactosylation of IVIg grouped by time.

FIG. 11 shows the effect of salt on IgG1 galactosylation by glycopeptide LCMS.

FIG. 12 shows the effect of salt on IgG1 sialylation by glycopeptide LCMS.

FIG. 13 shows the effect of salt on IgG2/3 galactosylation by glycopeptide LCMS.

FIG. 14 shows the effect of salt on IgG2/3 sialylation by glycopeptide LCMS.

FIG. 15 shows the effect of salt on IgG3/4 galactosylation by glycopeptide LCMS.

FIG. 16 shows the effect of salt on IgG3/4 sialylation by glycopeptide LCMS.

FIG. 17 shows a scheme for conversion of UDP-Gal to UMP and UDP.

FIG. 18 demonstrates non-specific degradation of UDP-Gal could be detected in the galactosylation of IVIg.

FIG. 19 demonstrates non-specific degradation of UDP-Gal could be detected in the galactosylation of IVIg.

### DETAILED DESCRIPTION

Antibodies are glycosylated at conserved positions in the constant regions of their heavy chain and on the Fab domain. For example, human IgG antibodies have a single N-linked glycosylation site at Asn297 of the CH2 domain. Each antibody isotype has a distinct variety of N-linked carbohydrate structures in the constant regions. For human IgG, the core oligosaccharide normally consists of GlcNAc₂Man₃GlcNAc, with differing numbers of outer residues. Variation among individual IgG's can occur via attachment of galactose and/or galactose-sialic acid at one or both terminal GlcNAc or via attachment of a third GlcNAc arm (bisecting GlcNAc).

The present disclosure encompasses, in part, methods for preparing immunoglobulins (e.g., human IgG) having an Fc region having particular levels of branched glycans that are sialylated on both of the arms of the branched glycan (e.g., with a NeuAc-α 2,6-Gal terminal linkage). The levels can be measured on an individual Fc region (e.g., the number of branched glycans that are sialylated on an α1,3 arm, an α1,6 arm, or both, of the branched glycans in the Fc region), or on the overall composition of a preparation of polypeptides (e.g., the number or percentage of branched glycans that are sialylated on an α1,3 arm, an α1,6 arm, or both, of the branched glycans in the Fc region in a preparation of polypeptides).

Naturally derived polypeptides that can be used to prepare hypersialylated IgG include, for example, IgG in human serum (particular human serum pooled from more than 1,000 donors), intravenous immunoglobulin (IVIg) and polypeptides derived from IVIg (e.g., polypeptides purified from IVIg (e.g., enriched for sialylated IgGs) or modified IVIg (e.g., IVIg IgGs enzymatically sialylated).

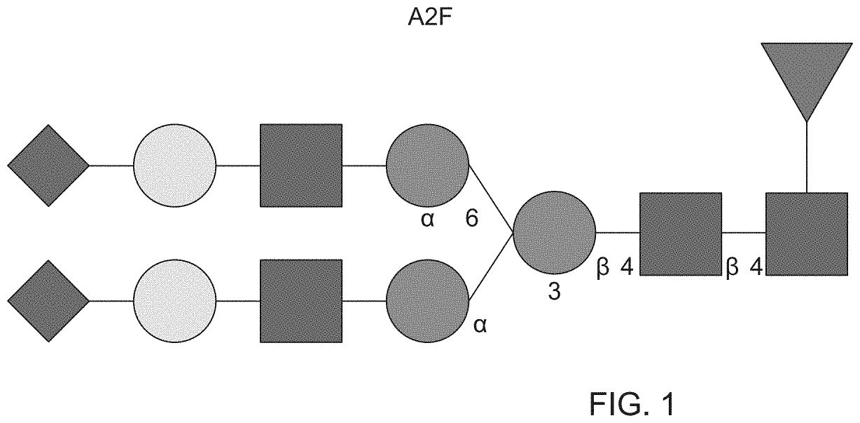

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α1,3 arm,” and the second branch is referred to as the “α1,6 arm,” as shown in FIG. 1.

N-glycans can be subdivided into three distinct groups called “high mannose type,” “hybrid type,” and “complex type,” with a common pentasaccharide core (Man (α1,6)-(Man(α1,3))-Man(β1,4)-GlcpNAc(β1,4)-GlcpNAc(β1,N)-Asn) occurring in all three groups.

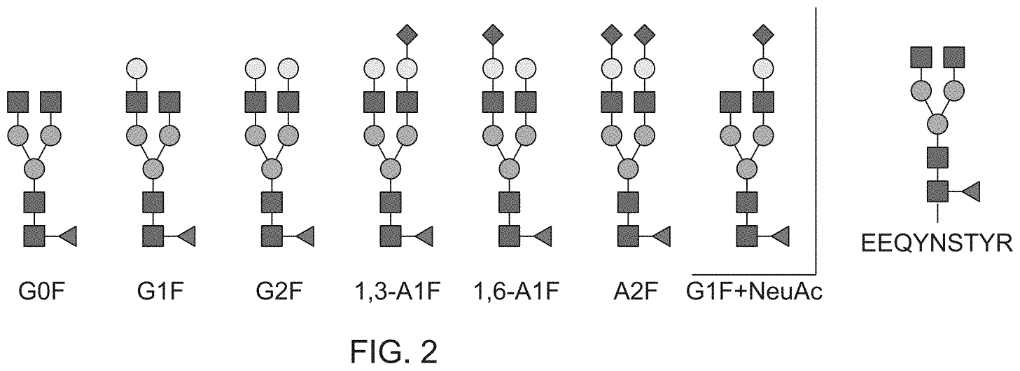

The more common Fc glycans present in IVIg are shown in FIG. 2.

Additionally or alternatively, one or more monosaccharides units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialic acid residues found in polypeptides produced in mammalian expression systems are N-acetylneuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialic acid groups can be either α 2,3 or α 2,6.

Fc regions are glycosylated at conserved, N-linked glycosylation sites. For example, each heavy chain of an IgG antibody has a single N-linked glycosylation site at Asn297 of the CH2 domain. IgA antibodies have N-linked glycosylation sites within the CH2 and CH3 domains, IgE antibodies have N-linked glycosylation sites within the CH3 domain, and IgM antibodies have N-linked glycosylation sites within the CH1, CH2, CH3, and CH4 domains.

Each antibody isotype has a distinct variety of N-linked carbohydrate structures in the constant regions. For example, IgG has a single N-linked biantennary carbohydrate at Asn297 of the CH2 domain in each Fc polypeptide of the Fc region, which also contains the binding sites for C1q and FcγR. For human IgG, the core oligosaccharide normally consists of GlcNAc2Man3GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc).

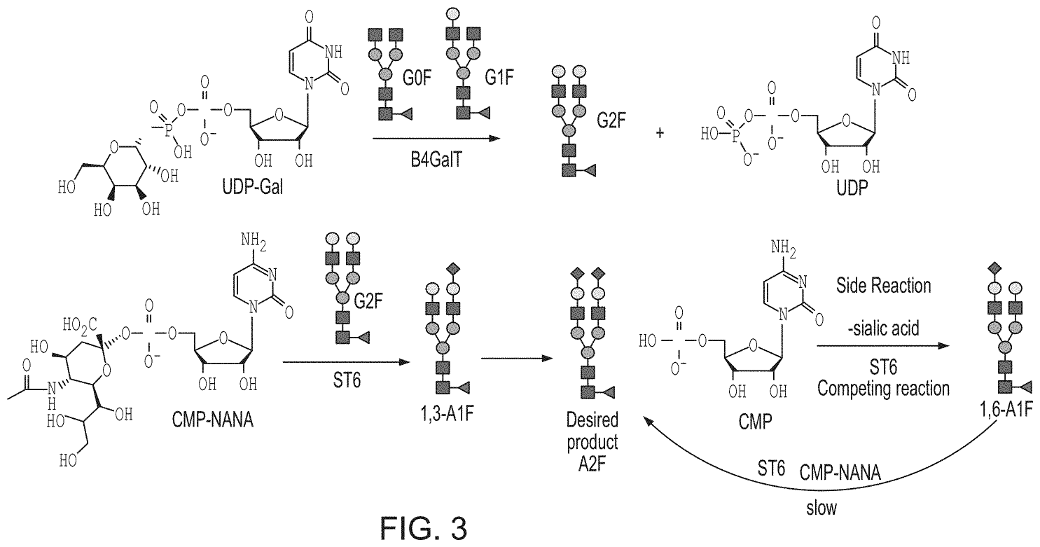

Immunoglobulins, e.g., IgG antibodies, can be sialylated by carrying out a galactosylation step followed by a sialylation step. Beta-1,4-galactosyltransferase 1 (B4GalT) is a Type II Golgi membrane-bound glycoprotein that transfers galactose from uridine 5′-diphosphategalactose ([[(2R,3S,4R,5R)-5-(2,4-dioxopyrimidin-1-yl)-3,4-dihydroxyoxolan-2-yl]methoxy-hydroxyphosphoryl] [(2R,3R,4S,5R,6R)-3,4,5-trihydroxy-6-(hydroxymethyl)oxan-2-yl] hydrogen phosphate; UDP-Gal) to GlcNAc as a β-1,4 linkage. Alpha-2,6-sialyltransferase 1 (ST6) is a Type II Golgi membrane-bound glycoprotein that transfers sialic acid from cytidine 5′-monophospho-Nacetylneuraminicacid ((2R,4S,5R,6R)-5-acetamido-2-[[(2R,3S,4R,5R)-5-(4-amino-2-oxopyrimidin-1-O-3,4-dihydroxyoxolan-2-yl]methoxy-hydroxyphosphoryl]oxy-4-hydroxy-6-(1,2,3-trihydroxypropyl)oxane-2-carboxylic acid; CMP-NANA or CMP-Sialic Acid) to Gal as an α-2,6 linkage. Schematically, the reactions proceed shown in FIG. 3.

Glycans of polypeptides can be evaluated using any methods known in the art. For example, sialylation of glycan compositions (e.g., level of branched glycans that are sialylated on an α1,3 branch and/or an α1,6 branch) can be characterized using methods described in WO2014/179601.

In some embodiments of the hsIgG compositions prepared by the methods described herein, at least 60%, 65%, 70%, 75%, 80%, 85%, or 90% of the branched glycans on the Fc domain have a sialic acid on both the α1,3 arm and the α1,6 arm that is connected through a NeuAc-α 2,6-Gal terminal linkage. In addition, in some embodiments, at least 40%, 50%, 60%, 65%, 70%, 75%, 80%, or 85% of the branched glycans on the Fab domain have a sialic acid on both the α1,3 arm and the α1,6 arm that is connected through a NeuAc-α 2,6-Gal terminal linkage. Overall, in some embodiments, at least 60%, 65%, 70%, 75%, 80%, 85%, or 90% of the branched glycans have a sialic acid on both the α1,3 arm and the α1,6 arm that is connected through a NeuAc-α 2,6-Gal terminal linkage.

In some embodiments, the hsIgG compositions prepared by the methods described herein comprises at least 50%, 55%, 60%, 65%, 70% or 75% of the branched glycans on the Fc domain have a sialic acid on both the α1,3 arm and the α1,6 arm.

### Enzymes

### Enzyme Activity

As described herein, 1 U B4GlaT B4GalT is equal to the formation of 1 nmol Gal-GlcNAc (also referred to as LacNAc) by the transfer of Gal from UDP-Gal and to GlcNAc per minute. Enzyme

As described herein, 1 U ST6Gal1 is equal to the formation of 1 nmol NeuAc-Gal-GlcNAc (also referred to as Sa-LacNAc) by the transfer of NeuAc from CMP-NANA and to Gal-GlcNAc (LacNAc) per minute.

### Galactosylating Enzymes

Beta-1,4-galactosyltransferase (B4GalT), e.g., human B4GalT, e.g., human B4Galt1, as well as orthologs, mutants, and variants thereof, including enzymatically active portions of beta-1,4-galactosyltransferase (B4GalT), e.g., human B4GalT, e.g., human B4Galt1, as well as orthologs, mutants, and variants thereof, along with fusion proteins and polypeptides comprising the same are suitable for use in the methods described herein. Beta-1,4-galactosyltransferase 1 (B4GalT) is a Type II Golgi membrane-bound glycoprotein that transfers galactose from uridine 5′-diphosphategalactose (UDP-Gal) to GlcNAc as a β-1,4 linkage. B4Galt1 is one of seven beta-1,4-galactosyltransferase (beta4GalT) genes that each encode type II membrane-bound glycoproteins that appear to have exclusive specificity for the donor substrate UDP-galactose; all transfer galactose in a beta1,4 linkage to similar acceptor sugars: GlcNAc, Glc, and Xyl. B4Galt1 adds galactose to N-acetylglucosamine residues that are either monosaccharides or the nonreducing ends of glycoprotein carbohydrate chains. B4GalT1 is also called GGTB2. Four alternative transcripts encoding four isoforms of B4GALT1 (NCBI Gene ID 2683) are described in Table 1.

TABLE 1
Human B4GALT1 isoforms

| Transcript | Length (nt) | Protein | SEQ ID NO: | Length (aa) | Isoform |
|---|---|---|---|---|---|
| NM_001497.4 | 4176 | NP_001488.2 | SEQ ID NO: 5 | 398 | 1 |
| NM_001378495.1 | 3999 | NP_001365424.1 | SEQ ID NO: 6 | 385 | 2 |
| NM_001378496.1 | 4053 | NP_001365425.1 | SEQ ID NO: 7 | 357 | 3 |
| NM_001378497.1 | 1520 | NP_001365426.1 | SEQ ID NO: 8 | 225 | 4 |

TABLE 2
Topology of B4GALT1 isoform 1 (SEQ ID NO: 5)

| | | Descrip- | | | SEQ ID |
|---|---|---|---|---|---|
| Feature | AAs | tion | Length | Sequence | NO: |
| Topological | 1-24 | Cytoplasmic | 9 | MRLREPLLSGSAAMPGASLQR | SEQ ID |
| domain | | | | ACR | NO: 9 |
| Transmembrane | 25-44 | Helical; | 17 | LLVAVCALHLGVTLVYYLAG | SEQ ID |
| | | Signal- | | | NO: 10 |
| | | anchor for | | | |
| | | type II | | | |
| | | membrane | | | |
| | | protein | | | |
| Topological | 45-398 | Lumenal | 380 | RDLSRLPQLVGVSTPLQGGSNS | SEQ ID |
| domain | | | | AAAIGQSSGELRTGGARPPPPL | NO: 11 |
| | | | | GASSQPRPGGDSSPVVDSGPGP | |
| | | | | ASNLTSVPVPHTTALSLPACPE | |
| | | | | ESPLLVGPMLIEFNMPVDLELV | |
| | | | | AKQNPNVKMGGRYAPRDCVSPH | |
| | | | | KVAIIIPFRNRQEHLKYWLYYL | |
| | | | | HPVLQRQQLDYGIYVINQAGDT | |
| | | | | IFNRAKLLNVGFQEALKDYDYT | |
| | | | | CFVFSDVDLIPMNDHNAYRCFS | |
| | | | | QPRHISVAMDKFGFSLPYVQYF | |
| | | | | GGVSALSKQQFLTINGFPNNYW | |
| | | | | GWGGEDDDIFNRLVFRGMSISR | |
| | | | | PNAVVGRCRMIRHSRDKKNEPN | |
| | | | | PQRFDRIAHTKETMLSDGLNSL | |
| | | | | TYQVLDVQRYPLYTQITVDIGT | |
| | | | | PS | |

TABLE 3
Binding sites of B4GALT1 isoform 1 (SEQ ID NO: 5)

| Position(s) | Description | Reference(s) |
|---|---|---|
| 250 | Metal binding; | |
| | Manganese | |
| 310 | Binding site; UDP- | “Structural snapshots of beta-1,4-galactosyltransferase-I along |
| | alpha-D-galactose | the kinetic pathway.” |
| | | Ramakrishnan B., Ramasamy V., Qasba P. K. |
| | | J. Mol. Biol. 357: 1619-1633(2006) |
| 343 | Metal binding; | |
| | Manganese; via | |
| | tele nitrogen | |
| 355 | Binding site; N- | “Oligosaccharide preferences of beta1,4-galactosyltransferase- |
| | acetyl-D- | I: crystal structures of Met340His mutant of human beta1,4- |
| | glucosamine | galactosyltransferase-I with a pentasaccharide and |
| | | trisaccharides of the N-glycan moiety.” |
| | | Ramasamy V., Ramakrishnan B., Boeggeman E., Ratner D. M., |
| | | Seeberger P. H., Qasba P. K. |
| | | J. Mol. Biol. 353: 53-67(2005) |
| | | “Deoxygenated disaccharide analogs as specific inhibitors of |
| | | beta1-4-galactosyltransferase 1 and selectin-mediated tumor |
| | | metastasis.” |
| | | Brown J. R., Yang F., Sinha A., Ramakrishnan B., Tor Y., |
| | | Qasba P. K., Esko J. D. |
| | | J. Biol. Chem. 284: 4952-4959(2009) |

TABLE 4
Post Translational Amino Acid Modifications of B4GALT1 isoform 1 (SEQ ID NO: 5)

| Feature key | Position(s) | Description | Reference(s) |
|---|---|---|---|
| Glycosylation | 113 | N-linked | |
| | | (GlcNAc . . .) | |
| | | asparagine | |
| Disulfide bond | 130 ↔ 172 | | “Oligosaccharide preferences of beta1,4- |
| Disulfide bond | 243 ↔ 262 | | galactosyltransferase-I: crystal structures of |
| | | | Met340His mutant of human beta1,4- |
| | | | galactosyltransferase-I with a pentasaccharide and |
| | | | trisaccharides of the N-glycan moiety.” |
| | | | Ramasamy V., Ramakrishnan B., Boeggeman E., |
| | | | Ratner D. M., Seeberger P. H., Qasba P. K. |
| | | | J. Mol. Biol. 353: 53-67(2005) |
| | | | “Structural snapshots of beta-1,4- |
| | | | galactosyltransferase-I along the kinetic pathway.” |
| | | | Ramakrishnan B., Ramasamy V., Qasba P. K. |
| | | | J. Mol. Biol. 357: 1619-1633(2006) |

The soluble form of B4GalT1 derives from the membrane form by proteolytic processing. The cleavage site is at positions 77-78 of B4GALT1 isoform 1 (SEQ ID NO: 5).

In some embodiments, one or more of the amino acids of the B4GalT1 corresponding to amino acids 113, 130, 172, 243, 250, 262, 310, 343, or 355 of B4GALT1 isoform 1 (SEQ ID NO: 5) is conserved as compared to (SEQ ID NO: 5).

In some embodiments, the enzyme is an enzymatically active portion of, e.g., B4GalT1. In some embodiments, the enzyme is an enzymatically active portion of B4GALT1 isoform 1 (SEQ ID NO: 5), or an ortholog, mutant, or variant of SEQ ID NO: 5. In some embodiments, the enzyme is an enzymatically active portion of B4GALT1 isoform 2 (SEQ ID NO: 6), or an ortholog, mutant, or variant of SEQ ID NO: 6. In some embodiments, the enzyme is an enzymatically active portion of B4GALT1 isoform 3 (SEQ ID NO: 7), or an ortholog, mutant, or variant of SEQ ID NO: 7. In some embodiments, the enzyme is an enzymatically active portion of B4GALT1 isoform 4 (SEQ ID NO: 8), or an ortholog, mutant, or variant of SEQ ID NO: 8.

In some embodiments, the enzymatically active portion of B4GalT1 does not comprise a cytoplasmic domain, e.g., SEQ ID NO: 9. In some embodiments, the enzymatically active portion of B4GalT1 does not comprise a transmembrane domain, e.g., SEQ ID NO: 10. In some embodiments, the enzymatically active portion of B4GalT1 does not comprise a cytoplasmic domain, e.g., SEQ ID NO: 9 or a transmembrane domain, e.g., SEQ ID NO: 10.

In some embodiments, the enzymatically active portion of B4GalT1 comprises all or a portion of a luminal domain, e.g., SEQ ID NO: 11, or an ortholog, mutants, or variants thereof.

In some embodiments, the enzymatically active portion of B4GalT1 comprises amino acids 109-398 of SEQ ID NO: 5, or an ortholog, mutants, or variants thereof. In some embodiments, the enzymatically active portion of B4GalT1 consists of SEQ ID NO: 5, or an ortholog, mutant, or variant of SEQ ID NO: 5.

A suitable functional portion of an B4GalT1 can comprise or consist of an amino acid sequence that is at least 80% (85%, 90%, 95%, 98% or 100%) identical to SEQ ID NO: 12.

Also suitable for use in the methods described herein is an amino acid sequence that comprises or consists of an amino acid sequence that is at least 80% (85%, 90%, 95%, 98% or 100%) identical to SEQ ID NO: 13.

### Silylating Enzymes

ST6, e.g., ST6Gal1, e.g., human ST6Gal1, as well as orthologs, mutants, and variants thereof, including enzymatically active portions of ST6Gal1, e.g., human ST6Gal1, as well as orthologs, mutants, and variants thereof, along with fusion proteins and polypeptides comprising the same, are suitable for use in the methods described herein. Alpha-2,6-sialyltransferase 1 (ST6) is a Type II Golgi membrane-bound glycoprotein that transfers sialic acid from cytidine 5′-monophospho-N-acetylneuraminic acid (CMP-NANA) to Gal as an α-2,6 linkage. ST6Gal1 is also called as ST6N or SIAT1. Four alternative transcripts encoding two isoforms of ST6GAL1 (NCBI Gene ID 6480) are described in Table 1.

TABLE 1
Human ST6GAL1 isoforms

| Transcript | Length (nt) | Protein | SEQ ID NO: | Length (aa) | Isoform |
|---|---|---|---|---|---|
| NM_173216.2 | 4604 | NP_775323.1 | SEQ ID NO: 14 | 406 | a |
| NM_173217.2 | 3947 | NP_775324.1 | SEQ ID NO: 15 | 175 | b |
| NM_003032.3 | 4303 | NP_003023.1 | SEQ ID NO: 14 | 406 | a |
| NM_001353916.2 | 4177 | NP_001340845.1 | SEQ ID NO: 14 | 406 | a |

TABLE 2
Topology of ST6Gal1 isoform a (SEQ ID NO: 14)

| | | Descrip- | | | SEQ ID |
|---|---|---|---|---|---|
| Feature | AAs | tion | Length | Sequence | NO: |
| Topological | 1-9 | Cytoplasmic | 9 | MIHTNLKKK | SEQ ID |
| domain | | | | | NO: 16 |
| Transmembrane | 10-26 | Helical; | 17 | FSCCVLVFLLFAVICVW | SEQ ID |
| | | Signal- | | | NO: 17 |
| | | anchor for | | | |
| | | type II | | | |
| | | membrane | | | |
| | | protein | | | |
| Topological | 27-406 | Lumenal | 380 | KEKKKGSYYDSFKLQTKEFQVLKSLGK | SEQ ID |
| domain | | | | LAMGSDSQSVSSSSTQDPHRGRQTLGS | NO: 18 |
| | | | | LRGLAKAKPEASFQVWNKDSSSKNLIP | |
| | | | | RLQKIWKNYLSMNKYKVSYKGPGPGIK | |
| | | | | FSAEALRCHLRDHVNVSMVEVTDFPFN | |
| | | | | TSEWEGYLPKESIRTKAGPWGRCAVVS | |
| | | | | SAGSLKSSQLGREIDDHDAVLRFNGAP | |
| | | | | TANFQQDVGTKTTIRLMNSQLVTTEKR | |
| | | | | FLKDSLYNEGILIVWDPSVYHSDIPKW | |
| | | | | YQNPDYNFFNNYKTYRKLHPNQPFYIL | |
| | | | | KPQMPWELWDILQEISPEEIQPNPPSS | |
| | | | | GMLGIIIMMTLCDQVDIYEFLPSKRKT | |
| | | | | DVCYYYQKFFDSACTMGAYHPLLYEKN | |
| | | | | LVKHLNQGTDEDIYLLGKATLPGFRTI | |
| | | | | HC | |

TABLE 3
Binding sites of ST6Gal1 isoform a (SEQ ID NO: 14)

| Position(s) | Description | Reference(s) |
|---|---|---|
| 189 | Substrate; via amide | “The structure of human alpha-2,6-sialyltransferase reveals |
| | nitrogen | the binding mode of complex glycans.” |
| 212 | Substrate | Kuhn B., Benz J., Greif M., Engel A. M., Sobek H., Rudolph |
| 233 | Substrate | M. G. Acta Crystallogr. D 69: 1826-1838(2013) |
| 353 | Substrate; via | |
| | carbonyl oxygen | |
| 354 | Substrate | |
| 365 | Substrate | |
| 369 | Substrate | |
| 370 | Substrate | “The structure of human alpha-2,6-sialyltransferase reveals |
| 376 | Substrate | the binding mode of complex glycans.” |
| | | Kuhn B., Benz J., Greif M., Engel A. M., Sobek H., Rudolph |
| | | M. G. Acta Crystallogr. D 69: 1826-1838(2013) |

TABLE 4
Post Translational Amino Acid Modifications of ST6Gal1 isoform a (SEQ ID NO: 14)

| Feature key | Position(s) | Description | Reference(s) |
|---|---|---|---|
| Disulfide | 142 ↔ 406 | | “The structure of human alpha-2,6- |
| bond | | | sialyltransferase reveals the binding mode of |
| | | | complex glycans.” |
| | | | Kuhn B., Benz J., Greif M., Engel A. M., Sobek |
| | | | H., Rudolph M. G. |
| | | | Acta Crystallogr. D 69: 1826-1838(2013) |
| Glycosylation | 149 | N-linked | “Glycoproteomics analysis of human liver tissue |
| | | (GlcNAc. . .) | by combination of multiple enzyme digestion and |
| | | asparagine | hydrazide chemistry.” |
| | | | Chen R., Jiang X., Sun D., Han G., Wang F., Ye |
| | | | M., Wang L., Zou H. |
| | | | J. Proteome Res. 8: 651-661(2009); and |
| | | | “The structure of human alpha-2,6- |
| | | | sialyltransferase reveals the binding mode of |
| | | | complex glycans.” |
| | | | Kuhn B., Benz J., Greif M., Engel A. M., Sobek |
| | | | H., Rudolph M. G. |
| | | | Acta Crystallogr. D 69: 1826-1838(2013) |
| Glycosylation | 161 | N-linked | “Glycoproteomics analysis of human liver tissue |
| | | (GlcNAc. . .) | by combination of multiple enzyme digestion and |
| | | asparagine | hydrazide chemistry.” |
| | | | Chen R., Jiang X., Sun D., Han G., Wang F., Ye |
| | | | M., Wang L., Zou H. |
| | | | J. Proteome Res. 8: 651-661(2009) |
| Disulfide | 184 ↔ 335 | | “The structure of human alpha-2,6- |
| bond | | | sialyltransferase reveals the binding mode of |
| | | | complex glycans.” |
| | | | Kuhn B., Benz J., Greif M., Engel A. M., Sobek |
| | | | H., Rudolph M. G. |
| | | | Acta Crystallogr. D 69: 1826-1838(2013) |
| Disulfide | 353 ↔ 364 | | “The structure of human alpha-2,6- |
| bond | | | sialyltransferase reveals the binding mode of |
| | | | complex glycans.” |
| | | | Kuhn B., Benz J., Greif M., Engel A. M., Sobek |
| | | | H., Rudolph M. G. |
| | | | Acta Crystallogr. D 69: 1826-1838(2013) |
| Modified | 369 | Phosphotyrosine | “Quantitative phosphoproteomic analysis of T |
| residue | | | cell receptor signaling reveals system-wide |
| | | | modulation of protein-protein interactions.” |
| | | | Mayya V., Lundgren D. H., Hwang S.-I., Rezaul |
| | | | K, Wu L., Eng J. K., Rodionov V., Han D. K. |
| | | | Sci. Signal. 2: RA46-RA46(2009) |

The soluble form of ST6Gal1 derives from the membrane form by proteolytic processing.

In some embodiments, one or more of the amino acids of the ST6Gal1 corresponding to amino acids 142, 149, 161, 184, 189, 212, 233, 335, 353, 354, 364, 365, 369, 370, 376, or 406 of ST6Gal1 isoform a (SEQ ID NO: 14) is conserved as compared to SEQ ID NO: 14.

Also provided herein is an enzymatically active portion of, e.g., ST6Gal1. In some embodiments, the enzyme is an enzymatically active portion of ST6Gal1 isoform a (SEQ ID NO: 14), or an ortholog, mutant, or variant of SEQ ID NO: 14. In some embodiments, the enzyme is an enzymatically active portion of ST6Gal1 isoform b (SEQ ID NO: 15), or an ortholog, mutant, or variant of SEQ ID NO: 15.

In some embodiments, the enzymatically active portion of ST6Gal1 does not comprise a cytoplasmic domain, e.g., SEQ ID NO: 16. In some embodiments, the enzymatically active portion of ST6Gal1 does not comprise a transmembrane domain, e.g., SEQ ID NO: 17. In some embodiments, the enzymatically active portion of ST6Gal1 does not comprise a cytoplasmic domain, e.g., SEQ ID NO: 16 or a transmembrane domain, e.g., SEQ ID NO: 17.

In some embodiments, the enzymatically active portion of ST6Gal1 comprises all or a portion of a luminal domain, e.g., SEQ ID NO: 18, or an ortholog, mutants, or variants thereof.

In some embodiments, the enzymatically active portion of ST6Gal1 comprises amino acids 87-406 of SEQ ID NO: 14 (SEQ ID NO: 19), or an ortholog, mutants, or variants thereof. In some embodiments, the enzymatically active portion of ST6Gal1 consists of SEQ ID NO: 19, or an ortholog, mutant, or variant of SEQ ID NO: 19.

A suitable functional portion of an ST6Gal1 can comprise or consist of an amino acid sequence that is at least 80% (85%, 90%, 95%, 98% or 100%) identical to SEQ ID NO: 19.

In some embodiments, the ST6Gal1 comprises or consists of SEQ ID NO: 19, the portion of SEQ ID NO: 19 from amino acid 4 to 320, or the portion of SEQ ID NO: 19 from amino acid 5 to 320.

Also suitable for use in the methods described herein is an amino acid sequence that comprises or consists of an amino acid sequence that is at least 80% (85%, 90%, 95%, 98% or 100%) identical to SEQ ID NO: 20.

### Antibodies

The methods described herein include galactosylation and sialylation of antibodies. Suitable antibodies include, for example, IgG antibodies. The antibodies, e.g., IgG antibodies, can be pooled. For example, pooled IgG antibodies include IVIg.

In some embodiments, the IgG antibodies comprise IgG antibodies isolated from at least 1000 donors.

In some embodiments, at least 50%, 55%, 60%, 65% or 70% w/w of the IgG antibodies are IgG1 antibodies.

In some embodiments, at least 90% of the donor subject has been exposed to a virus.

In some embodiments, the methods described herein include providing a mixture of IgG antibodies. In some embodiments, providing a mixture of IgG antibodies includes (a) providing pooled plasma from at least 1000 human subjects; and (b) isolating a mixture of IgG antibodies from the pooled plasma. In some embodiments, the mixture of IgG antibodies are isolated from intravenous immunoglobulin. In some embodiments, the mixture of IgG antibodies are intravenous immunoglobulin. In some embodiments, the step of isolating a mixture of IgG antibodies from the pooled plasma comprises ethanol precipitation or caprylic acid (also called octanoic acid) precipitation. In some embodiments, the step of isolating a mixture of IgG antibodies from the pooled plasma comprises biding IgG antibodies to an ion exchange column and eluting the IgG antibodies from an ion exchange column.

In some embodiments, the antibod(ies), e.g., the antibod(ies) described herein, e.g., IgG, e.g., pooled IgG, e.g., IVIg, is provided as part of a solution. In some embodiments, the concentration of antibod(ies), e.g., antibod(ies) described herein, e.g., pooled IgG, e.g., IVIg, is from or from about 100 mg/mL to or to about 200 mg/mL. In some embodiments, the concentration of the antibod(ies) is 150 mg/mL or is about 150 mg/mL.

In some embodiments, the solution consists of or comprises the antibod(ies), e.g., the antibod(ies) described herein, e.g., IgG, e.g., pooled IgG, e.g., IVIg, and a buffer. In some embodiments, the buffer is selected from the group consisting of BIS-TRIS, MOPS, MES, PIPES, BES, MOPSO, TEA, POPSO, EPPS, and combinations thereof.

In some embodiments, the solution consists of or comprises the antibod(ies), e.g., the antibod(ies) described herein, e.g., IgG, e.g., pooled IgG, e.g., IVIg, and BIS-TRIS buffer. In some embodiments, the solution consists of or comprises the antibod(ies) in 50 mM BIS-TRIS buffer.

In some embodiments, the solution consists of or comprises the antibod(ies), e.g., the antibod(ies) described herein, e.g., IgG, e.g., pooled IgG, e.g., IVIg, and BIS-TRIS buffer, e.g., 50 mM BIS-TRIS buffer, at from or from about pH 6.8 to or to about pH 7.4. In some embodiments, the solution consists of or comprises the antibod(ies), e.g., the antibod(ies) described herein, e.g., IgG, e.g., pooled IgG, e.g., IVIg, and BIS-TRIS buffer, e.g., 50 mM BIS-TRIS buffer at from or from about pH 6.8 to or to about pH 7.3, from or from about pH 6.8 to or to about pH 7.2, from or from about pH 6.8 to or to about pH 7.1, from or from about pH 6.8 to or to about pH 7.0, from or from about pH 6.8 to or to about pH 6.9, from or from about pH 6.9 to or to about pH 7.4, from or from about pH 7.3 to or to about pH 7.2, from or from about pH 6.9 to or to about pH 7.1, from or from about pH 6.9 to or to about pH 7.0, from or from about pH 7.0 to or to about pH 7.4, from or from about pH 7.0 to or to about pH 7.3, from or from about pH 7.0 to or to about pH 7.2, from or from about pH 7.0 to or to about pH 7.1, from or from about pH 7.1 to or to about pH 7.4, from or from about pH 7.1 to or to about pH 7.3, from or from about pH 7.1 to or to about pH 7.2, from or from about pH 7.2 to or to about pH 7.4, or from or from about pH 7.3 to or to about pH 7.4. In some embodiments, the solution consists of or comprises the antibod(ies), e.g., the antibod(ies) described herein, e.g., IgG, e.g., pooled IgG, e.g., IVIg, and BIS-TRIS buffer, e.g., 50 mM BIS-TRIS buffer at or at about pH 7.3.

### Enzymatic Galactosylation and Sialylation

The methods described herein can comprise a galactosylation step. An exemplary galactosylation reaction is depicted in FIG. 3. Thus, provided herein is a method for galactosylating antibod(ies), e.g., antibod(ies) described herein, by providing a composition (a galactosylation mixture) comprising: antibod(ies), e.g., antibod(ies) described herein; a galatosylating enzyme, e.g., a galatosylating enzyme described herein, e.g., B4GalT or enzymatically active portion of variant thereof; UDP-gal or salt thereof; a buffer, e.g., a buffer described herein, e.g., BIS-TRIS buffer; and optionally MnCl₂, and incubating the composition under conditions effective for galactosylating the antibody, e.g., as described herein, thereby producing galactosylated antibod(ies).

The methods described herein can comprise a sialylation step. An exemplary sialylation reaction is depicted in FIG. 3. Thus, provided herein is a method for sialylating, e.g., hyper-sialylating, antibod(ies), e.g., antibod(ies) described herein, by providing a composition (a sialylation reaction mixture) comprising: galactosylated antibod(ies), e.g., as described herein; a sialylating enzyme, e.g., a sialylating enzyme described herein, e.g., ST6Gal1 or enzymatically active portion or variant thereof; CMP-NANA or a salt thereof; a buffer, e.g., a buffer described herein, e.g., BIS-TRIS buffer; and optionally MnCl₂, and incubating the composition under conditions effective for sialylating the antibod(ies), e.g., as described herein.

In some embodiments, the galactosylation step and the sialylation step are carried out sequentially in the same reaction mixture, that is, the galactosylation reaction mixture becomes the sialylation reaction mixture upon addition of the sialylating enzyme and CMP-NANA or salt thereof. In some embodiments, there galactosylation reaction mixture is not filtered, fractionated, or purified prior to the sialylation step. In some embodiments, the galactosylation step and the sialylation step are carried out separately, e.g, pre-galactosylated antibod(ies) are provided, though they may have been processed (e.g., filtered, fractionated, or purified) and/or stored prior to the sialylation step.

Thus, the methods described herein can also comprise a sequential galactosylation and sialylation step. An exemplary galactosylation and sialylation reaction is depicted in FIG. 3. Thus, provided herein is a method for galactosylating and sialylating, e.g., hyper-sialylating, antibod(ies), e.g., antibod(ies) described herein, by a) providing a composition (a galactosylation reaction mixture) comprising: antibod(ies), e.g., as described herein; a galactosylating enzyme, e.g., a galactosylating enzyme described herein, e.g., B4GalT or enzymatically active portion or variant thereof; UDP-gal or a salt thereof; a buffer, e.g., a buffer described herein, e.g., BIS-TRIS buffer; and optionally MnCl₂; and b) incubating the composition under conditions effective for galactosylating the antibod(ies), e.g., as described herein; c) adding a sialylating enzyme, e.g., a sialylating enzyme described herein, e.g., ST6Gal1 or enzymatically active portion or variant thereof and CMP-NANA or salt thereof to the galactosylation reaction mixture, thereby producing a sialylation reaction mixture; and d) incubating the composition under conditions effective for sialylating the galactosylated antibod(ies), e.g., as described herein.

Also provided herein is a method for galactosylating and sialylating, e.g., hyper-sialylating antibod(ies), e.g., antibod(ies) described herein, by providing a composition comprising: antibod(ies), e.g., as described herein; a galactosylating enzyme, e.g., a galactosylating enzyme described herein, e.g., B4GalT or enzymatically active portion or variant thereof; UDP-gal or a salt thereof, and a buffer, e.g., a buffer described herein, e.g., BIS-TRIS buffer; a sialylating enzyme, e.g., a sialylating enzyme described herein, e.g., ST6Gal1 or enzymatically active portion or variant thereof; CMP-NANA or salt thereof, and optionally MnCl₂; and d) incubating the composition under conditions effective for galactosylating and sialylating the antibod(ies), e.g., as described herein.

In some embodiments, one or more component(s) of one or more of the reaction mixture(s) are supplemented during the incubation. That is, the reaction mixture may comprise an amount of the component at the beginning of the reaction (which may change during the course of the reaction), but also be supplemented with additional amounts of the component(s) during the reaction.

In some embodiments, the galactosylation reaction mixture comprises from or from about 50 to or to about 200 mg/mL antibod(ies), e.g., the antibod(ies) described herein, e.g., IgG, e.g., pooled IgG, e.g., IVIg. In some embodiments, the galactosylation reaction mixture comprises from or from about 50 to or to about 200, from or from about 50 to or to about 150, from or from about 50 to or to about 100, from or from about 100 to or to about 200, from or from about 100 to or to about 200, from or from about 100 to or to about 150, or from or from about 150 to or to about 200 mg/mL antibod(ies), e.g., the antibod(ies) described herein, e.g., IgG, e.g., pooled IgG, e.g., IVIg.

In some embodiments, the galactosylation reaction mixture comprises 50 mg/mL or more, 75 mg/mL or more, 100 mg/mL or more, 125 mg/mL or more, 150 mg/mL or more, or 200 mg/mL or more antibod(ies), e.g., the antibod(ies) described herein, e.g., IgG, e.g., pooled IgG, e.g., IVIg.

In some embodiments, the galactosylation reaction mixture comprises from or from about 6.0 to or to about 15.0 U galactosylating enzyme per gram of antibody. In some embodiments, the galactosylation reaction mixture comprises from or from about 7.0 to or to about 9.0 U galactosylating enzyme per gram of antibody. In some embodiments, the galactosylation reaction mixture comprises from or from about 7.2 to or to about 8.8 U of galactosylating enzyme per gram of antibody. In some embodiments, the galactosylation reaction mixture comprises 7.5 or about 7.5 U of galactosylating enzyme per gram of antibody. In some embodiments, the galactosylation reaction mixture comprises 8.0 or about 8.0 U of galactosylating enzyme per gram of antibody.

In some embodiments, the galactosylation reaction mixture is supplemented with from or from about 6.0 to or to about 15.0 U galactosylating enzyme per gram of antibody. In some embodiments, the galactosylation reaction mixture is supplemented with from or from about 7.0 to or to about 9.0 U galactosylating enzyme per gram of antibody. In some embodiments, the galactosylation reaction mixture is supplemented with from or from about 7.2 to or to about 8.8 U of galactosylating enzyme per gram of antibody. In some embodiments, the galactosylation reaction mixture is supplemented with 7.5 or about 7.5 U of galactosylating enzyme per gram of antibody. In some embodiments, the galactosylation reaction mixture is supplemented with 8.0 or about 8.0 U of galactosylating enzyme per gram of antibody.

In some embodiments, the galactosylation reaction mixture comprises from or from about 0.030 to or to about 0.050 mmol UDP-gal or salt thereof per gram of antibody. In some embodiments, the galactosylation mixture comprises from or from about 0.038 to or to about 0.046 mmol UDP-gal or salt thereof per gram of antibody. In some embodiments, the galactosylation reaction mixture comprises 0.038 or about 0.038 mmol UDP-gal or salt thereof per gram of antibody. In some embodiments, the galactosylation reaction mixture comprises 0.042 or about 0.042 mmol UDP-gal or salt thereof per gram of antibody.

In some embodiments, the galactosylation reaction mixture is supplemented with from or from about 0.030 to or to about 0.050 mmol UDP-gal or salt thereof per gram of antibody. In some embodiments, the galactosylation mixture is supplemented with from or from about 0.038 to or to about 0.046 mmol UDP-gal or salt thereof per gram of antibody. In some embodiments, the galactosylation reaction mixture is supplemented with 0.038 or about 0.038 mmol UDP-gal or salt thereof per gram of antibody. In some embodiments, the galactosylation reaction mixture is supplemented with 0.042 or about 0.042 mmol UDP-gal per gram of antibody.

In some embodiments, the sialylation reaction mixture comprises from or from about 14.0 to or to about 20.0 U sialylating enzyme per gram of antibody. In some embodiments, the sialylation reaction mixture comprises from or from about 17.1 to or to about 18.9 U of sialylating enzyme per gram of antibody. In some embodiments, the sialylation reaction mixture comprises 15.8 or about 15.8 U of sialylating enzyme per gram of antibody. In some embodiments, the sialylation reaction mixture comprises 18.0 or about 18.0 U of sialylating enzyme per gram of antibody.

In some embodiments, the sialylation reaction mixture is supplemented with from or from about 14.0 to or to about 20.0 U sialylating enzyme per gram of antibody. In some embodiments, the sialylation reaction mixture is supplemented with from or from about 17.1 to or to about 18.9 U of sialylating enzyme per gram of antibody. In some embodiments, the sialylation reaction mixture is supplemented with 15.8 or about 15.8 U of sialylating enzyme per gram of antibody. In some embodiments, the sialylation reaction mixture is supplemented with 18.0 or about 18.0 U of sialylating enzyme per gram of antibody.

In some embodiments, the sialylation reaction mixture comprises from or from about 0.1 to or to about 0.3 mmol CMP-NANA or salt thereof per gram of antibody. In some embodiments, the sialylation reaction mixture comprises from or from about 0.1425 to or to about 0.1575 mmol CMP-NANA or salt thereof per gram of antibody. In some embodiments, the sialylation reaction mixture comprises 0.220 or about 0.220 mmol CMP-NANA or salt thereof per gram of antibody. In some embodiments, the sialylation reaction mixture comprises 0.150 mmol or about 0.150 mmol CMP-NANA or salt thereof per gram of antibody.

In some embodiments, from or from about 0.01 to or to about 0.3 mmol CMP-NANA or salt thereof is added to the sialylation reaction mixture per gram of antibody at the beginning of the reaction. In some embodiments, from or from about 0.01425 to or to about 0.1575 mmol CMP-NANA or salt thereof is added to the sialylation reaction mixture per gram of antibody at the beginning of the reaction.

In some embodiments, the sialylation reaction mixture is supplemented with from or from about 0.01 to or to about 0.3 mmol CMP-NANA or salt thereof per gram of antibody. In some embodiments, the sialylation reaction mixture is supplemented with from or from about 0.01425 to or to about 0.1575 mmol CMP-NANA or salt thereof per gram of antibody.

In some embodiments, the total amount of CMP-NANA added to the sialylation reaction mixture is from or from about 0.1 to or to about 0.3 mmol CMP-NANA or salt thereof per gram of antibody. In some embodiments, the total amount of CMP-NANA added to the sialylation reaction mixture is from or from about 0.1425 to or to about 0.1575 mmol CMP-NANA or salt thereof per gram of antibody. In some embodiments, the total amount of CMP-NANA added to the sialylation reaction mixture is 0.220 or about 0.220 mmol CMP-NANA or salt thereof per gram of antibody. In some embodiments, the total amount of CMP-NANA added to the sialylation reaction mixture is 0.150 mmol or about 0.150 mmol CMP-NANA or salt thereof per gram of antibody.

In some embodiments, the sialylation reaction mixture is supplemented with CMP-NANA one, two, three, four, five, six, seven, eight, nine, or ten times. In some embodiments the sialylation mixture is supplemented with CMP-NANA less than seven times.

In some embodiments, the galactosylation and/or sialylation reaction mixture(s) each independently comprises from or from about 1 to or to about 20 mM MnCl₂. In some embodiments, the galactosylation and/or sialylation reaction mixture(s) each independently comprise from or from about 4.5 to or to about 5.5 mM MnCl₂. In some embodiments, the galactosylation and/or sialylation reaction mixture(s) each independently comprise 7.5 or about 7.5 mM MnCl₂. In some embodiments, the galactosylation and/or sialylation reaction mixture(s) each independently comprise 5.0 or about 5.0 mM MnCl₂.

In some embodiments, the galactosylation and/or sialylation reaction mixture(s) comprise BIS-TRIS buffer. In some embodiments, the galactosylation and/or sialylation reaction mixture(s) each independently comprise from or 10 to or to about 500 mM BIS-TRIS buffer. In some embodiments, the galactosylation and/or sialylation reaction mixture(s) each independently comprise from or from about 10 to or to about 400, from or from about 10 to or to about 300, from or from about 10 to or to about 300, from or from about 10 to or to about 200, from or from about 10 to or to about 100, from or from about 10 to or to about 50, from or from about 50 to or to about 500, from or from about 50 to or to about 400, from or from about 50 to or to about 300, from or from about 50 to or to about 200, from or from about 50 to or to about 100, from or from about 100 to or to about 500, from or from about 100 to or to about 400, from or from about 100 to or to about 300, from or from about 100 to or to about 200, from or from about 200 to or to about 500, from or from about 200 to or to about 400, from or from about 200 to or to about 300, from or from about 300 to or to about 500, from or from about 300 to or to about 400, or from or from about 400 to or to about 500 mM BIS-TRIS buffer. In some embodiments, the galactosylation and/or sialylation reaction mixture(s) each independently comprise from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 100, from or from about 80 to or to about 90, or from or from about 90 to or to about 100 mM BIS-TRIS buffer.

In some embodiments, the galactosylation and/or sialylation reaction mixture(s) each independently comprise 50 mM or less, 100 mM or less, 150 mM or less, 30 mM or less, or no sodium chloride.

In some embodiments, the buffer of the galactosylation and/or sialylation reaction mixture(s), e.g., the Bis-Tris buffer of the galactosylation and/or sialylation reaction mixture(s), each independently comprise 50 mM or less, 100 mM or less, 150 mM or less, 30 mM or less, or no sodium chloride.

In some embodiments, the galactosylation and/or sialylation steps are each independently carried out from or from about 20 to or to about 50° C. In some embodiments, the sialylation is carried out from or from about 20 to or to about 45, from or from about 20 to or to about 40, from or from about 20 to or to about 35, from or from about 20 to or to about 30, from or from about 20 to or to about 25, from or from about 25 to or to about 50, from or from about 25 to or to about 45, from or from about 25 to or to about 40, from or from about 25 to or to about 35, from or from about 25 to or to about 30, from or from about 30 to or to about 50, from or from about 30 to or to about 45, from or from about 30 to or to about 40, from or from about 30 to or to about 35, from or from about 35 to or to about 50, from or from about 35 to or to about 45, from or from about 35 to or to about 40, from or from about 40 to or to about 50, from or from about 40 to or to about 45, or from or from about 45 to or to about 50° C. In some embodiments, the sialylation is carried out at or at about 37° C.

In some embodiments, the galactosylation and/or sialylation steps are each independently carried out at our about pH 5.8 to or to about pH 7.2. In some embodiments, the sialylation is carried out at or at about pH 5.8 to or to about pH 7.1, at or at about pH 5.8 to or to about pH 7.0, at or at about pH 5.8 to or to about pH 6.9, at or at about pH 5.8 to or to about pH 6.8, at or at about pH 5.8 to or to about pH 6.7, at or at about pH 5.8 to or to about pH 6.6, at or at about pH 5.8 to or to about pH 6.5, at or at about pH 5.8 to or to about pH 6.4, at or at about pH 5.8 to or to about pH 6.3, at or at about pH 5.8 to or to about pH 6.2, at or at about pH 5.8 to or to about pH 6.1, at or at about pH 5.8 to or to about pH 6.0, at or at about pH 5.8 to or to about pH 5.9, at or at about pH 5.9 to or to about pH 7.2, at or at about pH 5.9 to or to about pH 7.1, at or at about pH 5.9 to or to about pH 7.0, at or at about pH 5.9 to or to about pH 6.9, at or at about pH 5.9 to or to about pH 6.8, at or at about pH 5.9 to or to about pH 6.7, at or at about pH 5.9 to or to about pH 6.6, at or at about pH 5.9 to or to about pH 6.5, at or at about pH 5.9 to or to about pH 6.4, at or at about pH 5.9 to or to about pH 6.3, at or at about pH 5.9 to or to about pH 6.2, at or at about pH 5.9 to or to about pH 6.0, at or at about pH 6.0 to or to about pH 7.2, at or at about pH 6.0 to or to about pH 7.1, at or at about pH 6.0 to or to about pH 7.0, at or at about pH 6.0 to or to about pH 6.9, at or at about pH 6.0 to or to about pH 6.8, at or at about pH 6.0 to or to about pH 6.7, at or at about pH 6.0 to or to about pH 6.6, at or at about pH 6.0 to or to about pH 6.5, at or at about pH 6.0 to or to about pH 6.4, at or at about pH 6.0 to or to about pH 6.3, at or at about pH 6.0 to or to about pH 6.2, at or at about pH 5.9 to or to about pH 6.1, at or at about pH 6.0 to or to about pH 7.2, at or at about pH 6.0 to or to about pH 7.1, at or at about pH 6.0 to or to about pH 7.0, at or at about pH 6.0 to or to about pH 6.9, at or at about pH 6.0 to or to about pH 6.8, at or at about pH 6.0 to or to about pH 6.7, at or at about pH 6.0 to or to about pH 6.6, at or at about pH 6.0 to or to about pH 6.5, at or at about pH 6.0 to or to about pH 6.4, at or at about pH 6.0 to or to about pH 6.3, at or at about pH 6.0 to or to about pH 6.2, at or at about pH 6.0 to or to about pH 6.1, at or at about pH 6.1 to or to about pH 7.2, at or at about pH 6.1 to or to about pH 7.1, at or at about pH 6.1 to or to about pH 7.0, at or at about pH 6.1 to or to about pH 6.9, at or at about pH 6.1 to or to about pH 6.8, at or at about pH 6.1 to or to about pH 6.7, at or at about pH 6.1 to or to about pH 6.6, at or at about pH 6.1 to or to about pH 6.5, at or at about pH 6.1 to or to about pH 6.4, at or at about pH 6.1 to or to about pH 6.3, at or at about pH 6.1 to or to about pH 6.2, at or at about pH 6.2 to or to about pH 7.2, at or at about pH 6.2 to or to about pH 7.1, at or at about pH 6.2 to or to about pH 7.0, at or at about pH 6.2 to or to about pH 6.9, at or at about pH 6.2 to or to about pH 6.8, at or at about pH 6.2 to or to about pH 6.7, at or at about pH 6.2 to or to about pH 6.6, at or at about pH 6.2 to or to about pH 6.5, at or at about pH 6.2 to or to about pH 6.4, at or at about pH 6.2 to or to about pH 6.3, at or at about pH 6.3 to or to about pH 7.2, at or at about pH 6.3 to or to about pH 7.1, at or at about pH 6.3 to or to about pH 7.0, at or at about pH 6.3 to or to about pH 6.9, at or at about pH 6.3 to or to about pH 6.8, at or at about pH 6.3 to or to about pH 6.7, at or at about pH 6.3 to or to about pH 6.6, at or at about pH 6.3 to or to about pH 6.5, at or at about pH 6.3 to or to about pH 6.4, at or at about pH 6.4 to or to about pH 7.2, at or at about pH 6.4 to or to about pH 7.1, at or at about pH 6.4 to or to about pH 7.0, at or at about pH 6.4 to or to about pH 6.9, at or at about pH 6.4 to or to about pH 6.8, at or at about pH 6.4 to or to about pH 6.7, at or at about pH 6.4 to or to about pH 6.6, at or at about pH 6.4 to or to about pH 6.5, at or at about pH 6.5 to or to about pH 7.2, at or at about pH 6.5 to or to about pH 7.1, at or at about pH 6.5 to or to about pH 7.0, at or at about pH 6.5 to or to about pH 6.9, at or at about pH 6.5 to or to about pH 6.8, at or at about pH 6.5 to or to about pH 6.7, at or at about pH 6.5 to or to about pH 6.6, at or at about pH 6.6 to or to about pH 7.2, at or at about pH 6.6 to or to about pH 7.1, at or at about pH 6.6 to or to about pH 7.0, at or at about pH 6.6 to or to about pH 6.9, at or at about pH 6.6 to or to about pH 6.8, at or at about pH 6.6 to or to about pH 6.7, at or at about pH 6.7 to or to about pH 7.2, at or at about pH 6.7 to or to about pH 7.1, at or at about pH 6.7 to or to about pH 7.0, at or at about pH 6.7 to or to about pH 6.9, at or at about pH 6.7 to or to about pH 6.8, at or at about pH 6.8 to or to about pH 7.2, at or at about pH 6.8 to or to about pH 7.1, at or at about pH 6.8 to or to about pH 7.0, at or at about pH 6.8 to or to about pH 6.9, at or at about pH 6.9 to or to about pH 7.2, at or at about pH 6.9 to or to about pH 7.1, at or at about pH 6.9 to or to about pH 7.0, at or at about pH 7.0 to or to about pH 7.2, at or at about pH 7.0 to or to about pH 7.1, or at or at about pH 7.1 to or to about pH 7.2.

In some embodiments, the pH of one or more of the reaction mixtures is adjusted during the galactosylation and/or sialylation, e.g., to fall within the preferred range, e.g., to return to or to about the starting pH.

In some embodiments, the galactosylation step is carried out for 60 hours or less, e.g., 50 hours or less, 40 hours or less, or preferably 20 hours or less or 20 hours or less.

In some embodiments, the galactosylation step is carried out for at least 8, 12, 18, 24, 30, or 40, but no more than 60 hours.

In some embodiments, the galactosylation step is carried out for or for about 8, 12, 18, 24, 30, 40, 50, or 60 hours.

In some embodiments, the sialylation step is carried out for 70 hours or less, e.g., 60 hour or less, 50 hours or less, or preferably 40 hours or less.

In some embodiments, the sialylation step is carried out for at least 8, 12, 18, 24, 30, 40, or 50 hours, but no more than 70 hours.

In some embodiments, the sialylation step is carried out for or for about 8, 12, 18, 24, 30, 40, 50, 60, or 70 hours.

In some embodiments, the total incubation time for galactosylation and sialylation, e.g., sequentially in the same reaction mixture, is 130 hours or less, e.g., 120 hours or less, 110 hours or less, 100 hours or less, 90 hours or less, 80 hours or less, preferably 70 hours or less or 60 hours or less.

In some embodiments, the total incubation time for galactosylation and sialylation, e.g., sequentially in the same reaction mixture, is at least 8, 12, 18, 24, 30, 40, 50, 60, 70, 80, 90, 100, or 120 hours, but no more than 130 hours.

In some embodiments, the total incubation time for galactosylation and sialylation, e.g., sequentially in the same reaction mixture, is or is about 8, 12, 18, 24, 30, 40, 50, 60, 70, 80, 90, 100, or 130 hours.

In some embodiments, at least or about 60%, 65%, 70%, 75%, 80%, or 85% of the branched glycans on the antibod(ies), e.g., hsIgG, have a sialic acid on both the α1,3 branch and the α1,6 branch.

In some embodiments, about or at least 60%, 65%, 70%, 75%, 80%, or 85% of the branched Fc glycans on the antibod(ies), e.g., hsIgG, have a sialic acid on both the α1,3 branch and the α1,6 branch.

In some embodiments, about or at least 60%, 65%, 70%, 75%, 80%, or 85% of the branched glycans on the Fab domain of the antibod(ies), e.g., hsIgG, have a sialic acid on both the α1,3 arm and the α1,6 arm that is connected through a NeuAc-α 2,6-Gal terminal linkage.

In some embodiments, about or at least 80% of the branched Fc glycans on the hsIgG have a sialic acid on both the α1,3 branch and the α1,6 branch.

In some embodiments, about or at least 60%, 65%, 70% of the branched glycans on the Fab domain of the antibod(ies), e.g., hsIgG, have a sialic acid on both the α1,3 arm and the α1,6 arm that is connected through a NeuAc-α 2,6-Gal terminal linkage.

In some embodiments, about or at least 85% of the of the branched Fc glycans on the antibod(ies), e.g., hsIgG, have a sialic acid on both the α1,3 branch and the α1,6 branch.

In some embodiments, about or at least 60%, 65%, 70% of the branched glycans on the Fab domain of the antibod(ies), e.g., hsIgG, have a sialic acid on both the α1,3 arm and the α1,6 arm that is connected through a NeuAc-α 2,6-Gal terminal linkage.

In some embodiments, about or at least 90% of the of the branched Fc glycans on the antibod(ies), e.g., hsIgG, have a sialic acid on both the α1,3 branch and the α1,6 branch.

In some embodiments, about or at least 60%, 65%, 70% of the branched glycans on the Fab domain of the antibod(ies), e.g., hsIgG, have a sialic acid on both the α1,3 arm and the α1,6 arm that is connected through a NeuAc-α 2,6-Gal terminal linkage.

An exemplary galactosylation and sialylation reaction is shown in the table below.

| | | | | Total Amount | | |
|---|---|---|---|---|---|---|
| | | | | Added to | | |
| | | Initial | | Reaction | | Incubation |
| Stage | Component | Concentration | | Mixture | | Time |
| Galactosylation | Pooled IgG, e.g., IVIg | 125 | mg/mL | 21 | g | ≤60 hours, e.g., |
| | in 50 mM BIS-TRIS, | | | | | ≤50 hours, |
| | adjusted to about | | | | | ≤40 hours, |
| | pH 7.3 | | | | | ≤30 hours or |
| | BIS-TRIS | 50 | mM | 2.4 | mL | ≤20 hours; |
| | MnCl₂ | 5.0 ± | | 0.735 | mL | preferably 24 |
| | | 0.5 mM | | | | or about |
| | B4GalT enzyme | 8.0 ± | | 0.390 | mL | 24 hours |
| | | 0.8 U/g | | | | |
| | | IVIg | | | | |
| | UDP-gal | 0.042 ± | | 0.880 | mL | |
| | | 0.004 mmol/g | | | | |
| | | IVIg | | | | |
| Sialylation | ST6 enzyme | 18.0 ± | | 4.13 | mL | ≤70 hours, e.g., |
| | | 0.9 U/g | | | | ≤60 hours, |
| | | IVIg | | | | ≤50 hours, or |
| | BIS-TRIS | 50 | mM | 5.7 | mL | ≤40 hours; |

| | | | | |
|---|---|---|---|---|
| CMP-NANA | 0.150 ± | 1.58 | mL | preferably |
| | 0.0075 mmol/g | | | 32 hours |
| | IVIg | | | or about |
| | | | | 32 hours |

### EXAMPLES

The invention is further described in the following examples, which do not limit the scope of the invention described in the claims.

### Example 1: Hypersialylated IgG Preparation

IgG in which more than 60% of the overall branched glycans are disialylated can be prepared as follows.

Briefly, a mixture of IgG antibodies is exposed to a sequential enzymatic reaction using β1,4 galactosyltransferase 1 (B4GalT) and α2,6-sialyltransferase (ST6Gal1) enzymes. The B4GalT does not need to be removed from the reaction before addition of ST6Gal1 and no partial or complete purification of the product is needed between the enzymatic reactions.

The galactosyltransferase enzyme selectively adds galactose residues to pre-existing asparagine-linked glycans. The resulting galactosylated glycans serve as substrates to the sialic acid transferase enzyme which selectively adds sialic acid residues to cap the asparagine-linked glycan structures attached to. Thus, the overall sialylation reaction employed two sugar nucleotides (uridine 5′-diphosphogalactose (UDP-Gal) and cytidine-5′-monophospho-N-acetylneuraminic acid (CMP-NANA)). The latter is replenished periodically to increase disialylated product relative to monosialylated product. The reaction includes the co-factor manganese chloride.

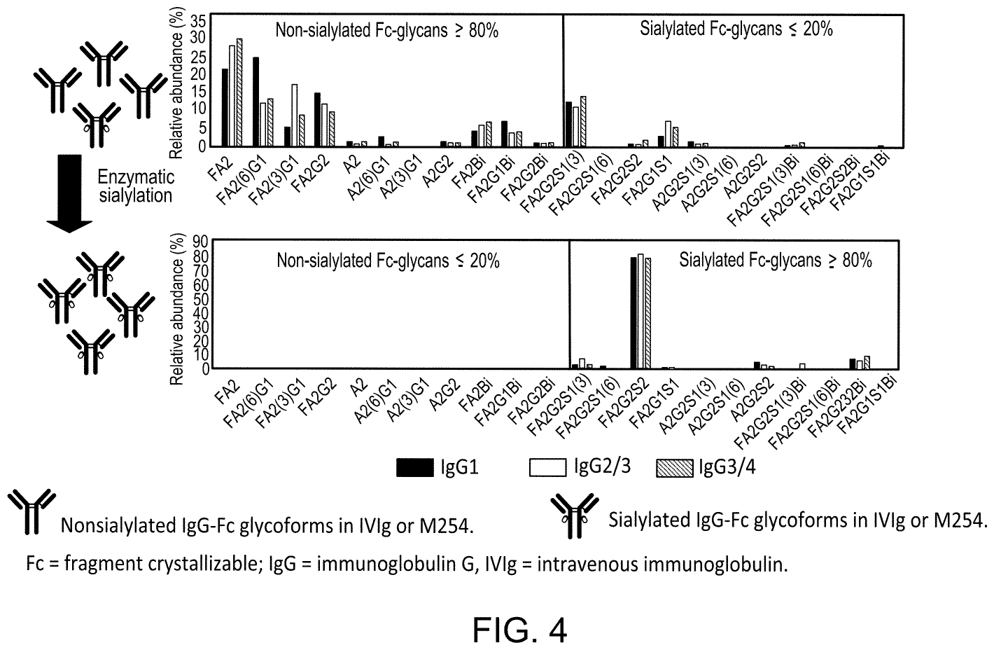

A representative example of the IgG-Fc glycan profile for such a reaction starting with IVIg and the reaction product is shown in the FIG. 4. In FIG. 4, the left panel is a schematic representation of enzymatic sialylation reaction to transform IgG to hsIgG; the right panel is the IgG Fc glycan profile for the starting IVIg and hsIgG. In this study, glycan profiles for the different IgG subclasses are derived via glycopeptide mass spectrometry analysis. The peptide sequences used to quantify glycopeptides for different IgG subclasses were: IgG1=EEQYNSTYR (SEQ ID NO: 1), IgG2/3 EEQFNSTFR (SEQ ID NO: 2), IgG3/4 EEQYNSTFR (SEQ ID NO: 3) and EEQFNSTYR (SEQ ID NO: 4).

The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is <20%. For the reaction product, the sum for all nonsialylated glycans is <20% and the sum for all sialylated glycans is more than 80%. Nomenclature for different glycans listed in the glycoprofile use the Oxford notation for N linked glycans.

### Example 2: Improvement of Sialylation Reaction

A wide ranging analysis of reactions conditions was carried out in an effort to further improve disialylation of IgG antibodies, including disialylation of Fc domain branched glycans. The sialylation reaction driven by ST6Gal using CMP-NANA as a substrate has characteristics that make improvement of the reaction, whether assessed by overall level of disialylation, time to reach a certain level of disialylation, amount of enzyme and substrate required to reach a certain overall level of disialylation, challenging. For example: (a) CMP-NANA is not entirely stable and will spontaneously hydrolyze even in the absence of any enzyme; (b) ST6Gal1 is thought to catalyze hydrolysis of the CMP-NANA without productive addition to the Gal on a branched glycan; (c) cytidine monophosphate (CMP), a side-product generated either through enzymatic addition or CMP-NANA hydrolysis, can act as a competitive inhibitor of ST6Gal1; (d) CMP has been observed to catalyze the reverse enzymatic reaction to remove the NeuAc from the newly formed glycan. Thus, over time the level of side-products will increase and this can lead to slowing or even reversal of the desired sialylation reaction.

There are reaction conditions that result in IgG antibodies or IVIg or pooled immune globulins with high levels of disialylation on Fc domain branched glycans. For example, sialylation with ST6Gal1 in MOPS buffer at pH 7.4 at 37° C. at a relatively high IgG antibodies concentration (e.g, a high concentration of IVIg concentration (≥125 mg/mL)) can provide high level disialylation of branched glycans, e.g., branched glycans on the Fc domain, when the sialylation reaction is carried out for a sufficiently long time and CMP-NANA is supplemented over the course of the sialylation reaction. Still, it would be desirable to find alternatives that reduce the use or concentration of substrates, reduce the reaction time or provide other improvements the production of hsIgG.

The addition of alkaline phosphatase to remove the phosphate from CMP and convert it to non-inhibitory cytidine was considered promising based on the nature of the sialylation reaction. However, this modification provided only a modest benefit under the conditions studied. Variation in the pH of the MOPS buffer did not appear to provide a meaningful benefit under the conditions studied. The addition of various metal ions was also explored, but did not appear to provide a meaningful benefit under the conditions studied. However, in the course of examining these variations it was observed that the addition of TRIS buffer, even as a co-buffer seemed to provide some benefit. In addition, under some conditions, it was observed that reduced CMP-NANA concentrations could be beneficial.

A variety of buffers with some structural similarity to TRIS and with various pKa were explored as an addition to or alternative for MOPS. Among the buffers tested are those in Table 5, below. Sialylation was carried out with ST6Gal1 and CMP-NANA on a Fc containing protein.

TABLE 5
Tested Buffers

| Buffer and pH | Buffer Name | Structure |
|---|---|---|
| 50 mM MOPS, pH 7.0 | 3-(N- morpholino)propanesulfonic acid | 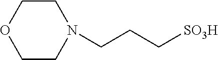 [SMILES: O=S(=O)(O)CCCN1CCOCC1] |
| 50 mM MES, pH 6.5 | 2-(N-morpholino)ethanesulfonic acid | 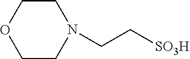 [SMILES: O=S(=O)(O)CCN1CCOCC1] |
| 50 mM BIS- TRIS pH 6.9 | Bis(2- hydroxyethyl)amino0tris (hydroxymethyl)methane | 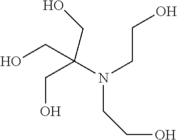 [SMILES: OCCN(CCO)C(CO)(CO)CO] |
| 50 mM PIPES, pH 7.0 | 1,4-Piperazinediethanesulfonic acid | 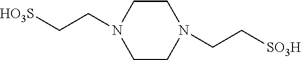 [SMILES: O=S(=O)(O)CCN1CCN(CCS(=O)(=O)O)CC1] |
| 50 mM BES, pH 7.0 | N,N-Bis(2-hydroxyethyl)-2- aminoethanesulfonic acid | 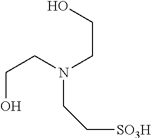 [SMILES: O=S(=O)(O)CCN(CCO)CCO] |
| 50 mM MOPSO, pH 7.1 | 3-morpholino-2- hydroxypropanesulfonic acid | 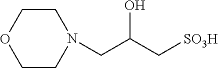 [SMILES: O=S(=O)(O)CC(O)CN1CCOCC1] |
| 50 mM TEA, pH 7.5 | Triethanolamine | 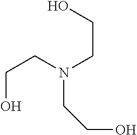 |
| 50 mM POPSO, pH 7.6 | Piperazine-N-N′-bis(2- hydroxypropanesulfonic acid | 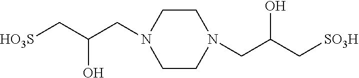 [SMILES: O=S(=O)(O)CC(O)CN1CCN(CC(O)CS(=O)(=O)O)CC1] |
| 50 mM POPSO, pH 8.0 | Piperazine-N,N′- bis(20hydroxypropanesulfonic acid | 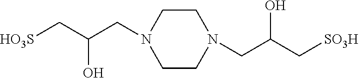 [SMILES: O=S(=O)(O)CC(O)CN1CCN(CC(O)CS(=O)(=O)O)CC1] |
| 50 mM TEA, pH 8.0 | Triethanolamine | 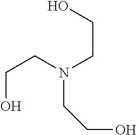 |
| 50 mM EPPS, pH 8.0 | 4-(2-Hydroxyethyl)-1- piperazinepropanesulfonic acid | 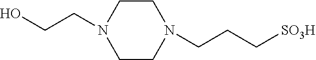 [SMILES: O=S(=O)(O)CCCN1CCN(CCO)CC1] |

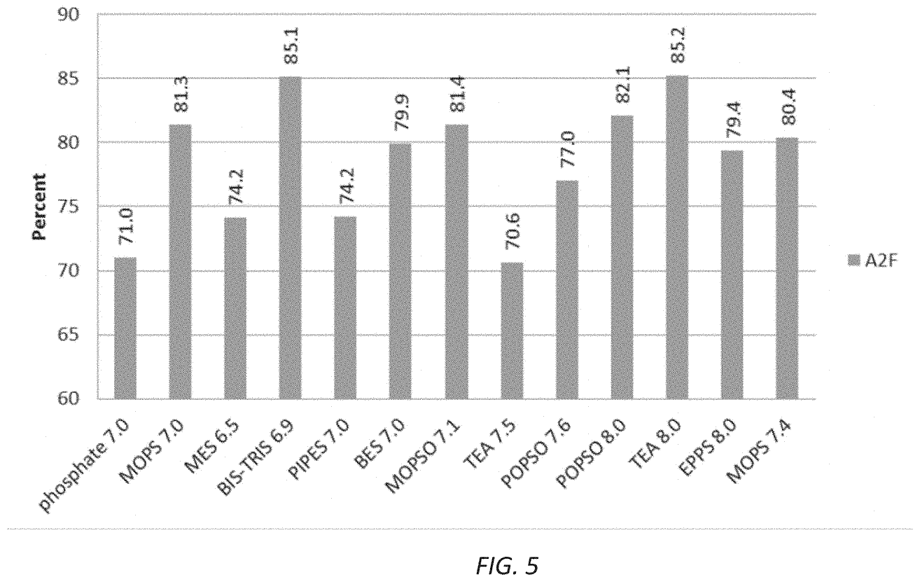

As can be seen in FIG. 5, the nature of the buffer impacted the level of A2F (71-85%).

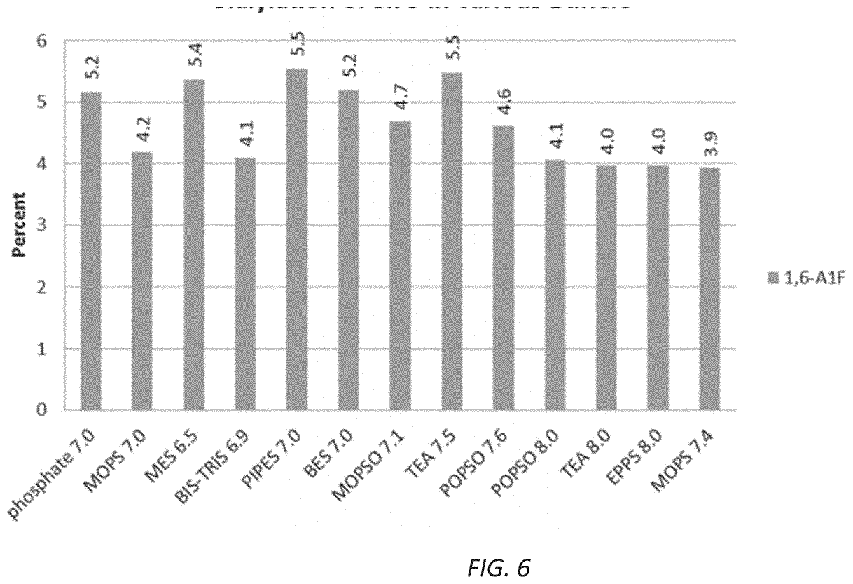

The formation of 1,6-A1F varies to a lesser extent across the buffers under the conditions examined, as shown in FIG. 6.

The sialylation of IVIg was investigated in BIS-TRIS pH 6.9, TEA pH 7.5, TEA pH 8.0, and TRIS pH 8.0. In addition, because it can be desirable to use the same buffer for both galactosylation and sialylation, for example, to provide a one pot reaction, the impact of the various buffers on galactosylation was also investigated. It was observed that certain buffers that appeared to be beneficial for sialylation were detrimental to galactosylation.

BIS-TRIS was selected for further study. Because some of the studies described above seemed to indicate that, under some conditions, reduced CMP-NANA could be beneficial, an extensive examination of enzyme and sugar nucleotide excess and dosing regimen was undertaken using IVIg as a substrate. As part of these studies, the impact of BIS-TRIS buffer on galactosylation was examined.

In the galactosylation of IVIg in BIS-TRIS pH 6.9 it was found that 50% less B4GalT enzyme could be used than when using MOPS pH 7.4. If the same amount of UDP-Gal was used, the galactosylation was complete in 15 h or less. For the subsequent sialylation reaction BIS-TRIS pH 6.9 it was found that it was possible to reduce the total amount of CMP-NANA by 50% and dramatically reduce the reaction time (from 72+ hours to 32-33 hours). Tables 6 and 7 below provide examples of the improvements observed in BIS-TRIS buffer.

TABLE 6
Galactosylation of IVIg Comparing MOPS to BIS-TRIS

| | | mU | Nmol UDP- | | |
|---|---|---|---|---|---|
| | | B4GalT/mg | Gal/mg | Galactosylation | G2F |
| Scale | Buffer | IVIg | IVIg | time (h) | (%) |
| 100 mg | 50 mM | 15 | 38 | 64 | 98 |
| | MOPS | | | | |
| | pH 7.4 | | | | |
| 2 mg | 50 mM | 7.8 | 39 | 24 | 99 |
| | BIS-TRIS | | | | |
| | pH 6.9 | | | | |

TABLE 7
Sialylation of IVIg Comparing MOPS to BIS-TRIS

| | | | | Total | Number | | |
|---|---|---|---|---|---|---|---|
| | | | mU | nmol CMP- | of CMP- | | |
| | | | ST6/mg | NANA/ | NANA | Time | A2F |
| Scale | | Buffer | IVIg | mg IVIg | additions | (h) | (%) |
| 15.5 | g | 50 mM | 14.9 | 470 | 7 | 88 | 91 |
| | | MOPS | | | | | |
| | | pH 7.4 | | | | | |
| 4.3 | mg | 50 mM | 14.2 | 221 | 2 | 32 | 95 |
| | | BIS-TRIS | | | | | |
| | | pH 6.9 | | | | | |
| 2.0 | g | 50 mM | 15.0 | 220 | 2 | 33 | 90 |
| | | BIS-TRIS | | | | | |
| | | pH 6.9 | | | | | |

Overall it was found that by changing from MOPS pH 7.4 to BIS-TRIS pH 6.9 is was possible to use less enzyme and/or less sugar nucleotides, yet achieve high level sialylation in considerably less time. Thus, suitable reaction conditions in 50 mM BIS-TRIS pH 6.9 include: galactosylation of IgG antibodies (e.g., pooled IgG antibodies, pooled immunoglobulins or IVIg) are as follows: 7.4 mM MnCl₂; 38 μmol UDP-Gal/g IgG antibody; and 7.5 units B4GalT/g IgG antibody with 16-24 hours of incubation at 37° C. followed by sialylation in 7.4 mM MnCl₂; 220 μmol CMP-NANA/g IgG antibody (added twice: half at the start of the reaction and half after 9-10 hrs); and 15 units ST6Gal1/g IgG antibody with 30-33 hours of incubation at 37°. The reaction can be carried out by adding the ST6Gal1 and CMP-NANA to the galactosylation reaction. Alternatively, all of the reactants can be combined at the outset and the CMP-NANA supplemented.

### Example 3: Enzymatic Galactosylation and Sialylation for Production of hsIgGs at High Concentration IVIg or IgG Antibodies

Sialylation is accomplished in two sequential enzymatic reaction steps using UDP-Gal and CMP-NANA in 50 mM MOPS buffer pH 7.4. Galactosylation occurs by reaction of IVIg (at about 135 mg/ml) with 8 to 15 units B4GalT/g IVIg and 0.038-0.042 mmol UDP-Gal/g IVIg in 50 mM MOPS buffer pH 7.4 with 5 to 8 mM MnCl₂. The reaction is allowed to proceed for 46 to 50 hrs and 37° C. Next, 15.8 to 18 units ST6Gal/g IVIg and CMP-NANA are added to the reaction and the concentration of IVIg is adjusted to about 120 mg/ml with 50 mM MOPS buffer pH 7.4. CMP-NANA is added at the outset of the sialylation reaction and is added 5 additional times at 8 to 12 hr intervals over a total 70-74 hrs reaction time at 37° C. The amount of CMP-NANA added is 400 μmol CMP-NANA/g IVIg. Thus, each addition is ⅙^{th} of the total amount added.

The reaction is then cooled to ambient temperature and diluted with 5× sodium phosphate buffer (PBS) 1:1 v/v.

Total glycans were assessed for sialylation. Greater than 97% of the glycans were sialylated and greater than 90% of the glycans were disialylated.

### Example 4: Reaction Conditions

The galactosylation reaction in the production of hsIgGs is relatively straightforward. However, the sialylation reaction has several challenges. First CMP-NANA is not stable and will spontaneously hydrolyze even in the absence of any enzyme. Additionally, ST6 enzyme is thought to also catalyze hydrolysis of the CMP-NANA without productive addition to the glycan acceptor. Cytidine monophosphate (CMP) is a side-product, generated either through enzymatic addition or CMP-NANA hydrolysis. CMP acts as a competitive inhibitor of ST6. Also, CMP has been observed to catalyze the reverse enzymatic reaction to remove the NeuAc from the newly formed glycan. Thus over time the concentration of CMP-NANA decreases, while as the side-products build up, the sialylation reaction slows and reverses. However, this reverse reaction appears to be far less favorable with BIS-TRIS as buffer when compared to MOPS as buffer.

M254 is currently produced by the enzymatic sialylation of Fc and Fab glycans of IVIg drug product in MOPS pH 7.4 buffer, all steps at 37° C., and using a very high IVIg concentration (˜150 mg/mL) where high protein concentration improves the reaction kinetics. The galactosylation step uses incubation over the course of 48 h. To compensate for the above discussed sialylation issues, a single addition of ST6 is followed by twice daily additions of CMP-NANA (six total) over the course of 72 h. This has been dubbed Process 2.0.

It is desirable to switch to an alternate process performed in BIS-TRIS buffer nominally at pH 6.90 which had been called Process 3.0.0. Process 3.0.0 uses less B4GalT enzyme, less CMP-NANA, and a shorter overall reaction time for both steps 56 h total vs. 120 h. Thus Process 3.0.0 incurs both lower materials cost and lower production costs.

The sialylation of IVIg in BIS-TRIS buffer using Process 3.0.0 has been carried out at both a lab scale (≤2 g) and at larger scales of 50 g and 250 g, respectively, at each of two different facilities. The disialylation extent obtained at the larger scales using BIS-TRIS buffer (Process 3.0.0) was lower than that observed at a lab scale and also lower than reactions done in MOPS buffer (Process 2.0) at the same facilities, though it still met the specification of ≥80% disialylation (Table 8). Therefore, a study was undertaken to try and understand what reaction conditions most influence this difference. Though the galactosylation step was shown to be near ideal it is recommended that the amounts of both UDP-Gal and B4GalT be increase by 10% to provide a cushion to assure the best results.

Additionally is was shown that less 30% CMP-NANA and 10% more ST6 relative to the initially used BIS-TRIS conditions could result in higher sialylation with minimal overall increase in materials cost.

TABLE 8
Disialylation as measured by TP-01167 for M254 at CMOs

| | | | Scale | Disialylation |
|---|---|---|---|---|
| IVIg | Buffer | Facility | (g) | (%) |
| Privigen | MOPS | 1 | 50 | 92.7 |
| Privigen | BIS-TRIS | 1 | 250, run 1 | 87.9 |
| Privigen | BIS-TRIS | 1 | 250, run 2 | 86.6 |
| ADMA | MOPS | 2 | 50 | 94.7 |
| ADMA | BIS-TRIS | 2 | 50 | 86.2 |
| Privigen | MOPS | 2 | 1500 | 92.7¹ |

¹Average of five GMP runs

### IVIg Solutions

IVIg solutions were prepared using Privigen IVIg drug product buffer exchanged into BIS-TRIS buffer. One batch of IVIg was buffer exchanged using a G25 desalting column equilibrated with BIS-TRIS pH 6.9 buffer followed by concentration of the IVIg flow thru fraction using a 10 kDa Vivaspin Turbo 15 device. Three 5 g batches of Privigen IVIg were buffer exchanged by tangential flow filtration (TFF) into 50 mM BIS-TRIS at pH 6.67, 6.93, and 7.11. One batch of IVIg was buffer exchanged by tangential flow filtration (TFF) into 50 mM BIS-TRIS at pH 6.9, followed by concentration using a 10 kDa Vivaspin Turbo 15 device.

IVIg lots used, method of buffer exchange, pH of buffer exchange, and measured final pH after concentration are shown in Table 9.

TABLE 9
IVIg solutions used

| | | pH of | Measured | |
|---|---|---|---|---|
| IVIg | Buffer | exchange | pH after | Concentration |
| lot | exchange method | buffer | concentration | (mg/mL) |
| 1 | G25/Vivaspin | 6.90 | 7.28 | 123 |
| | concentration | | | |
| 2 | TFF | 6.67 | 7.02 | 120 |
| 3 | TFF | 6.93 | 7.26 | 129 |
| 4 | TFF | 7.11 | 7.49 | 126 |
| 5 | TFF | 6.90 | 6.99 and 6.86^{a} | 135 |
| 6 | TFF then Vivaspin | 6.90 | 6.88 | 123 |
| | concentration | | | |

If, after buffer exchange and concentration, the pH of the IVIg solution lies outside of the preferred range, e.g., 7.2 to 7.4, preferably about 7.3, it should be adjusted.

### General Reaction Description

In general galactosylation was started first thing in the morning. After gentle mixing of reagents (IVIg, UDP-Gal, B4GalT enzyme, and MnCl₂) reactions were incubated at 37° C. without stirring or agitation. Reactions were not sterile filtered. Incubation was continued for various times. Two 5 uL aliquots were removed at various times and then frozen until analyzed. At the conclusion of an experiment the bulk reaction material was placed at 4° C.

The sialylation step was initiated (typically after 24 h galactosylation) by addition of ST6 enzyme and ½ the required CMP-NANA. In some cases the galactosylated material was first divide into smaller volumes to run multiple sialylation reactions. At 9 h the second ½ CMP-NANA was added. Incubation was continued for various times. Two 5 uL aliquots were removed at various times and then frozen until analyzed. At the conclusion of an experiment the bulk reaction material was placed at 4° C.

### Extent of Glycosylation

The extent of glycosylation was quantified by LCMS on the Fc glycopeptides. Table 10 shows the glycans quantified by LCMS on the Fc glycopeptides. Fully galactosylated encompassed all glycans having two galactose residues whether sialylated or not. Disialylation was defined as the sum of A2F, A2F+bisecting GlcNAc, and A2.

TABLE 10
Glycans quantified by LCMS on the Fc glycopeptides.

| | Included in fully | Included in |
|---|---|---|
| Glycans quantified | galactosylated | disialylated |
| G0F | — | — |
| G1F | — | — |
| G2F | G2F | — |
| 1,3-A1F | 1,3-A1F | — |
| 1,6-A1F | 1,6-A1F | — |
| A2F | A2F | A2F |
| G1F + NeuAc | — | — |
| G0F + bisect GlcNAc | — | — |
| G1F + bisect GlcNAc | — | — |
| G2F + bisect GlcNAc | G2F + bisect GlcNAc | — |
| A1F + bisect GlcNAc | A1F + bisect GlcNAc | — |
| A2F + bisect GlcNAc | A2F + bisect GlcNAc | A2F + bisect GlcNAc |
| G0 | — | — |
| G1 | — | — |
| G2 | — | — |
| 1,3-A1F | G2 | — |
| 1,6-A1F | 1,3-A1F | — |
| A2 | A2 | A2 |
| G1 + NeuAc | — | — |

### MnCl₂ Concentration

Prior to the start of this work an IVIg sialylation experiment was preformed where the amount of MnCl₂ was varied over a wide range (Error! Reference source not found.). ^{Error! Reference source not found.} This clearly indicated that high amounts of MnCl₂ was detrimental and also hinted that there might be different effects even around the 7.5 mM used in Process 3.0.0.

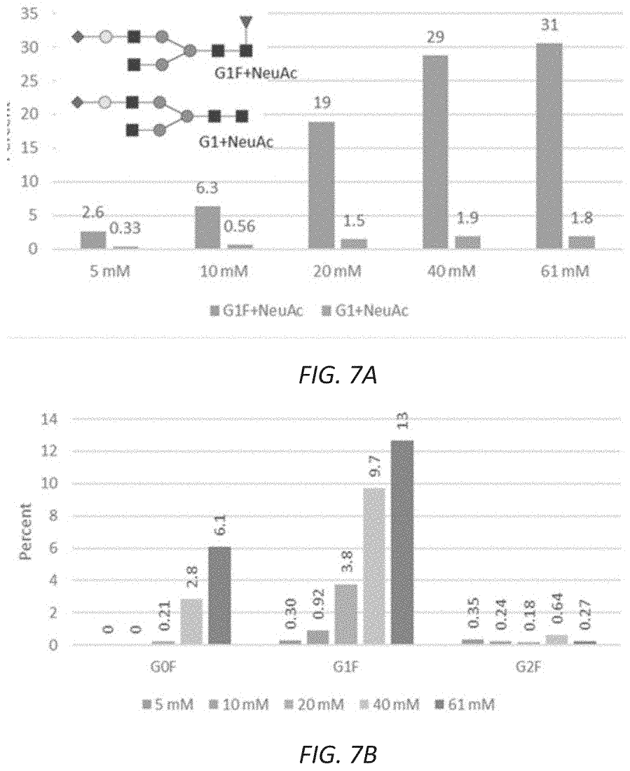

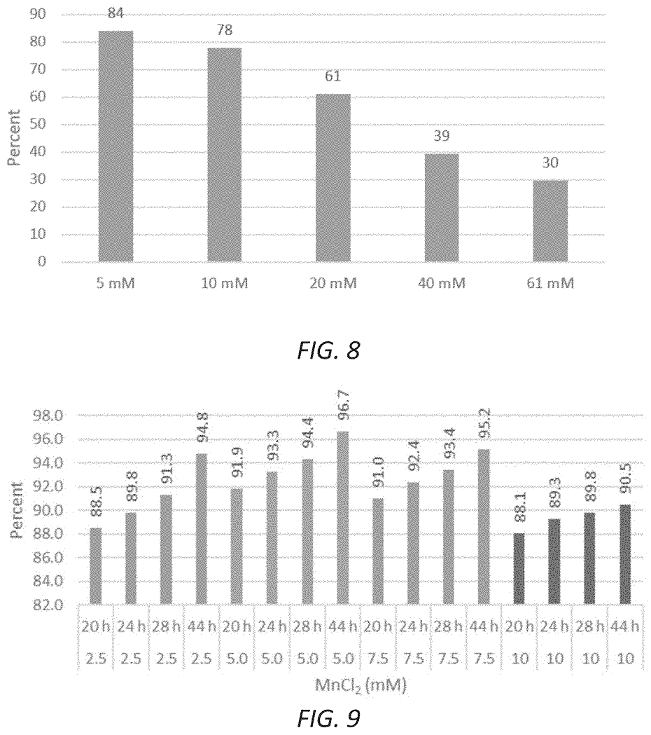

FIG. 7A shows the amount of G1F+NeuAc and G1+NeuAc increase with increasing MnCl₂. These species result from incomplete galactosylation. FIG. 7B shows that the amounts of G0F, G1F, and G2F increase with increasing MnCl₂. This shows that in addition to poorer galactosylation, sialylation is also affected, i.e. the amount of non-sialylated species is also higher with higher MnCl₂. FIG. 8 Error! Reference source not found. reiterates these results showing the disialylation level.

These results prompted additional experiments to look at the low end of MnCl₂ concentration from 2.5 mM to 10 mM. ^{Error! Reference source not found.} This set of reactions used IVIg buffer exchanged using G25 desalting and Vivaspin concentrators.

Galactosylation (using B4GalT and UDP-Gal Table 11) was performed and samples were removed for glycopeptide analysis after 20, 24, 28, and 44 h. The 44 h sample was then treated with CMP-NANA and ST6 for an additional 48 h with samples taken for analysis at 28, 32, 36, and 48 h which will be referred to as experimental series A. Separately, a set of samples was galactosylated for 24 h and then sialylated for another 48 h taking timed aliquots which will be referred to as experimental series B. LCMS glycopeptide data was analyzed using Qual Browser.

TABLE 11
Reagents used in MnCl₂ concentration investigation JS1169

| Reagent | Lot |
|---|---|
| MnCl₂ | SLBR8810V |
| IVIg pH 6.90 | 4323400386 (JS1164B) |
| B4GalT | 30716921 |
| UDP-Gal | combined lots 11840262, |
| | 11840277, 11840264 |
| | in MOPS |
| CMP-NANA | 37304121 |
| ST6 | 36071123 |

FIG. 9 shows an increase in galactosylation of IgG1 between 20 and 44 h for all conditions (grouped by MnCl₂ concentration). Similar results were seen for the other IgG subclasses.

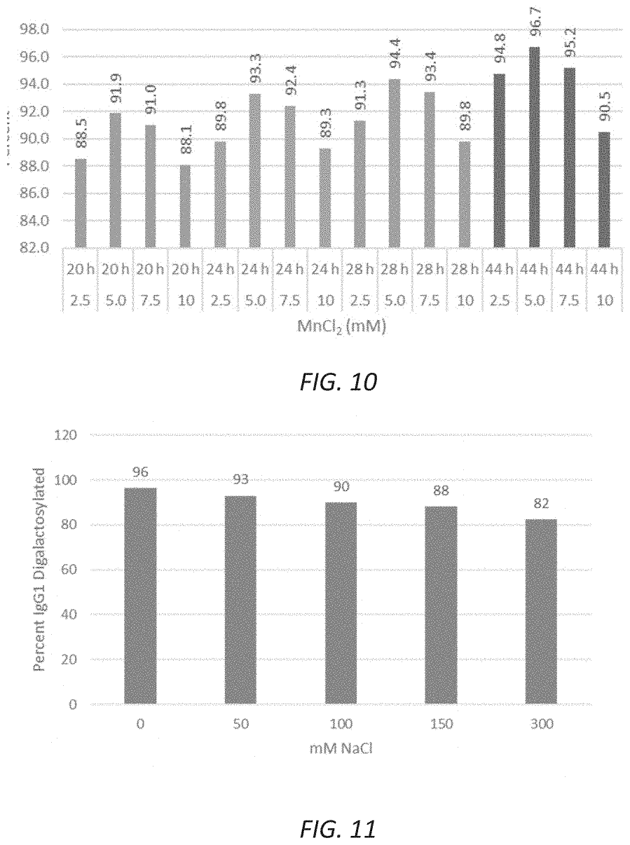

FIG. 10 shows the same data but grouped by time. Here one can see that at any one time galactosylation extent increases from 2.5 mM MnCl₂ to 5.0 mM and then falls going to 7.5 mM and then 10 mM. This clearly demonstrates that galactosylation with 5.0 mM MnCl₂ is better than the 7.5 mM MnCl₂ used in Process 3.0.0.

### Salt Concentration

IVIg in pH 6.9 buffer was subjected to the galactosylation and sialylation reaction using UDP-Gal/B4GalT and CMP-NANA/ST6 respectively in the presence of MnCl₂ with 37° C. incubation. Various concentrations of sodium chloride in BIS-TRIS buffer were added to obtain a final reaction concentration of 0, 50, 100, 150, and 300 mM sodium chloride. Samples were removed for glycan analysis by glycopeptide LCMS at the end of the galactosylation and sialylation reactions.

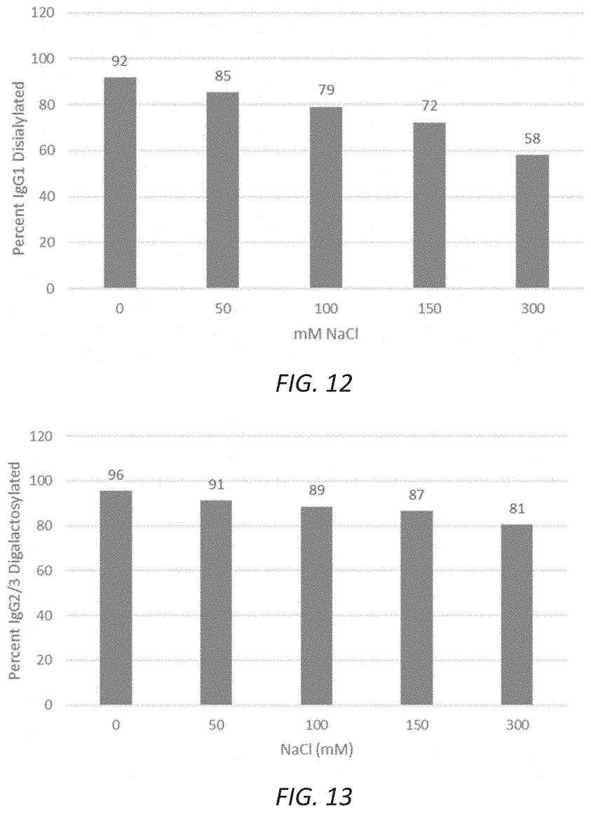

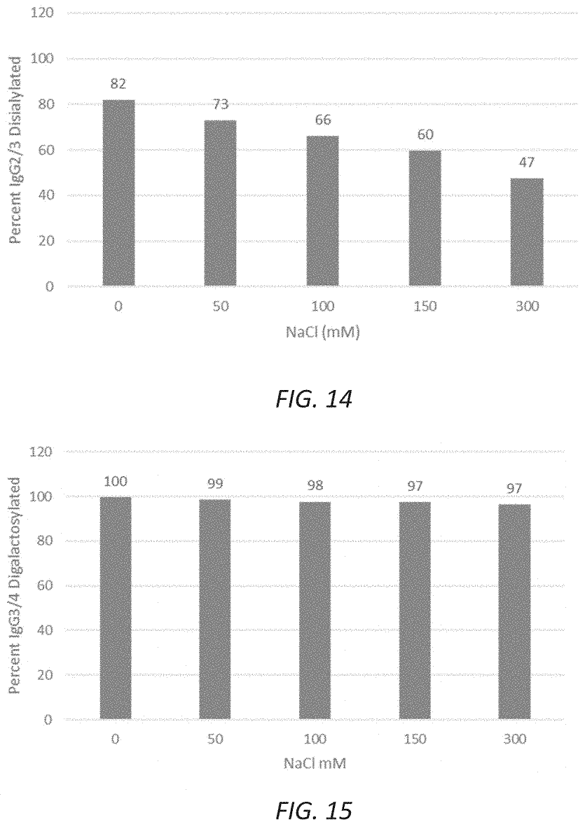

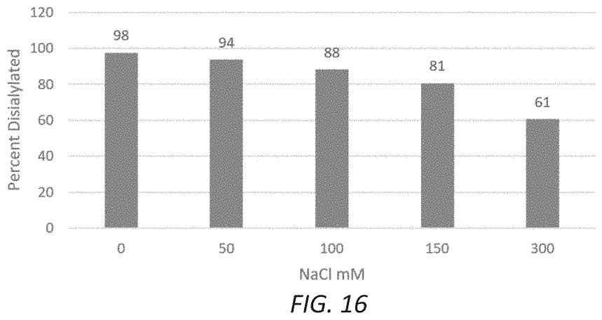

As shown in FIG. 11, FIG. 12, FIG. 13, FIG. 14, FIG. 15, and FIG. 16, addition of sodium chloride to the galactosylation and sialylation reactions had a negative effect on reaction extent in a salt concentration dependent manner. The effect was seen for all IgG subclasses and was most pronounced for the sialylation step. The presence of 50 mM sodium chloride could depress the sialylation extent by 4-9% depending on IgG subclass.

### UDP-Gal Stability

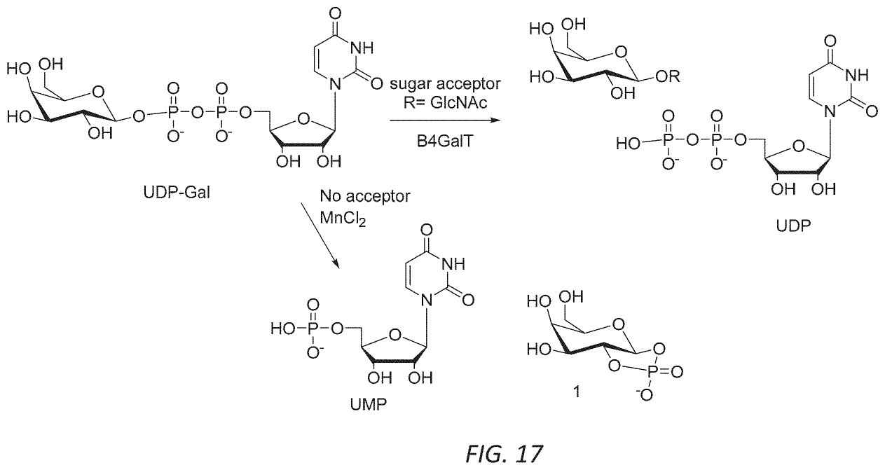

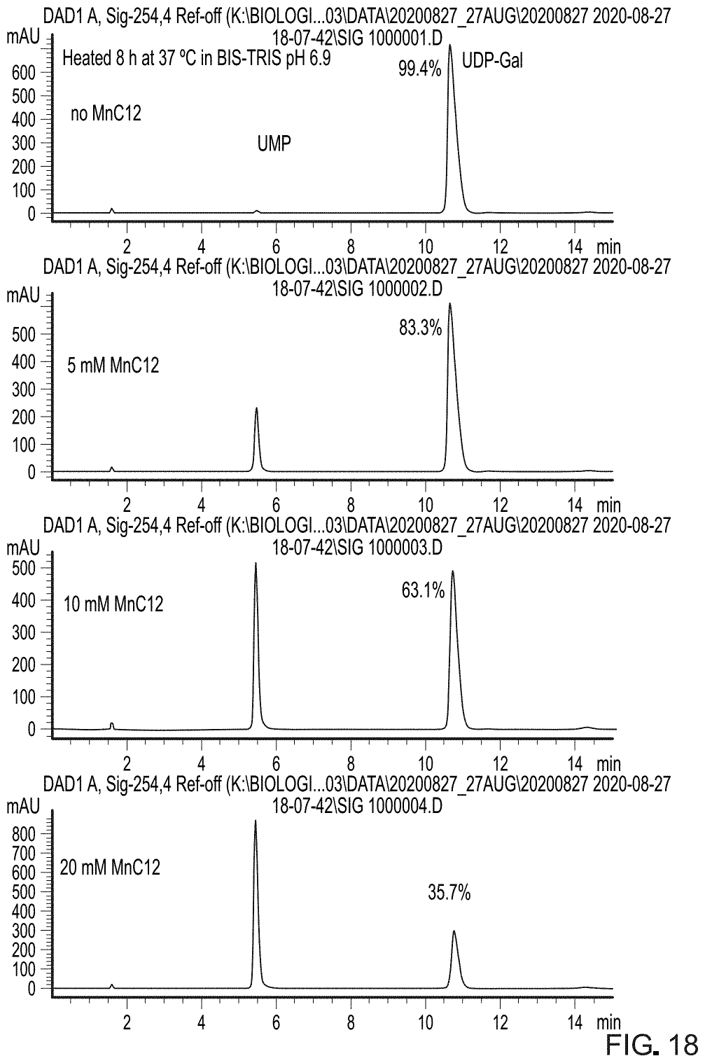

UDP-Gal was found to be unstable in the presence of MnCl₂ to give decomposition products (UMP and presumably 1,2-phosphogalactose 1) different from enzyme catalyzed transfer products (UDP). The mechanism is thought to follow the scheme shown in FIG. 17.

The amount of UDP-Gal, UDP, and UMP was assessed by ion pairing HPLC on a Supelcosil LC-18-T column using a 0.1 M potassium phosphate, 4 mM tetrabutylammonium bisulfate, pH 6.0 mobile phase and UV detection at 254 nm. Only components bearing uridine were detected by UV and sugars not bound to uridine could not be detected. Products were compared to known standards.

UDP-Gal in BIS-TRIS pH 6.9 buffer was heated at 37° C. in the presence of 0, 5, 10 or 20 mM MnCl₂ for 8 h and then assessed by ion pairing HPLC. No B4Galt was included in this experiment. The amount of UDP-Gal loss was MnCl₂ dependent and increased with increasing MnCl₂ concentration. The only other product observed was UMP. Very little UMP was visible in the absence of MnCl₂.

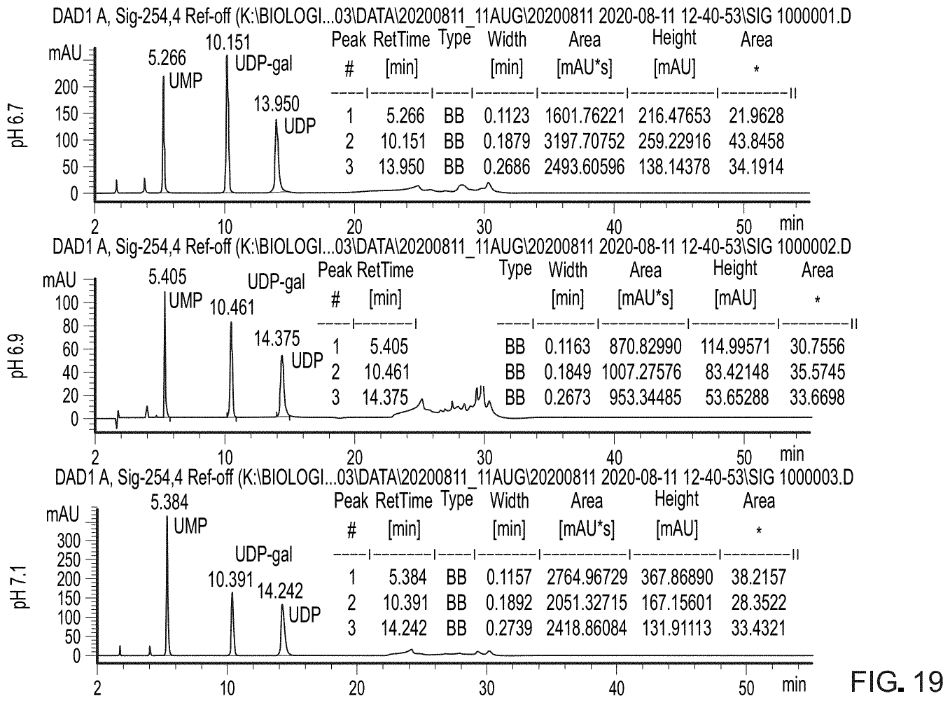

As shown in FIG. 18 and FIG. 19, this non-specific degradation of UDP-Gal could be detected in the galactosylation of IVIg. IVIg was galactosylated 24 h using UDP-Gal, B4GalT, and 5 mM MnCl₂ in BIS-TRIS buffer at three different pH (6.7, 6.9, and 7.1). The higher molecular weight IgG protein was separated from the low molecular weight sugar nucleotide using a 500 MWCO spin unit and the nucleotide containing fraction was injected onto the ion pairing HPLC. Formation of both UMP (peak 1) and UDP (peak 3) was visible, the UMP from the non-specific degradation and the UDP from enzyme catalyzed transfer to the glycans of IgG. Formation of UMP increased as the pH was changed from 6.7 to 6.9 to 7.1. Formation of UDP did not appear to be influenced by the pH range examined here.

### Example 5: Hypersialylated IgG Preparation

In another example, hsIgG is prepared using BIS-TRIS pH 7.3. Thus, suitable reaction conditions in 50 mM BIS-TRIS pH 7.3 include: galactosylation of IgG antibodies (e.g., pooled IgG antibodies, pooled immunoglobulins or IVIg) are as follows: 5.0 mM MnCl₂; 42 μmol UDP-Gal/g IgG antibody; and 8.0 units B4GalT/g IgG antibody with 16-24 hours of incubation at 37° C. followed by sialylation in 5.0 mM MnCl₂; 110 μmol CMP-NANA/g IgG antibody (added twice: half at the start of the reaction and again after 9-10 hrs); and 18 units ST6Gal1/g IgG antibody with 30-33 hours of incubation at 37°. The reaction can be carried out by adding the ST6Gal1 and CMP-NANA to the galactosylation reaction.

This method, carried out at a 21 g scale, achieved 99% full IgG1 galactosylation by glycopeptide LCMS, 96% disialylated IgG1 by glycopeptide LCMS, and 94% disialylated by N-glycan release (AdvanceBio Gly-X N-glycan prep with InstantPC kit, Agilent). This gives the global release of N-glycans allowing the quantitative sum of IgG1, IgG2, IgG3, IgG4 Fc glycans as well as the ˜15-25% Fab glycosylation present in IVIg.

### SEQUENCES

SEQ ID NO: 1
(IgG1)
EEQYNSTYR
SEQ ID NO: 2
(IgG2/3)
EEQFNSTFR
SEQ ID NO: 3
(IgG3/4)
EEQYNSTFR
SEQ ID NO: 4
(IgG3/4)
EEQFNSTYR
(NP_001488.2 B4GALT1
[organism = Homo sapiens]
[GeneID = 2683]
[isoform = 1])
SEQ ID NO: 5
MRLREPLLSGSAAMPGASLQRACRLLVAVCAL
HLGVTLVYYLAGRDLSRLPQLVGVSTPLQGGS
NSAAAIGQSSGELRTGGARPPPPLGASSQPRP
GGDSSPVVDSGPGPASNLTSVPVPHTTALSLP
ACPEESPLLVGPMLIEFNMPVDLELVAKQNPN
VKMGGRYAPRDCVSPHKVAIIIPFRNRQEHLK
YWLYYLHPVLQRQQLDYGIYVINQAGDTIFNR
AKLLNVGFQEALKDYDYTCFVFSDVDLIPMND
HNAYRCFSQPRHISVAMDKFGFSLPYVQYFGG
VSALSKQQFLTINGFPNNYWGWGGEDDDIFNR
LVFRGMSISRPNAVVGRCRMIRHSRDKKNEPN
PQRFDRIAHTKETMLSDGLNSLTYQVLDVQRY
PLYTQITVDIGTPS
(NP_001365424.1 B4GALT1
[organism = Homo sapiens]
[GeneID = 2683]
[isoform = 2])
SEQ ID NO: 6
MPGASLQRACRLLVAVCALHLGVTLVYYLAGR
DLSRLPQLVGVSTPLQGGSNSAAAIGQSSGEL
RTGGARPPPPLGASSQPRPGGDSSPVVDSGPG
PASNLTSVPVPHTTALSLPACPEESPLLVGPM
LIEFNMPVDLELVAKQNPNVKMGGRYAPRDCV
SPHKVAIIIPFRNRQEHLKYWLYYLHPVLQRQ
QLDYGIYVINQAGDTIFNRAKLLNVGFQEALK
DYDYTCFVFSDVDLIPMNDHNAYRCFSQPRHI
SVAMDKFGFSLPYVQYFGGVSALSKQQFLTIN
GFPNNYWGWGGEDDDIFNRLVFRGMSISRPNA
VVGRCRMIRHSRDKKNEPNPQRFDRIAHTKET
MLSDGLNSLTYQVLDVQRYPLYTQITVDIGTP
S
(NP_001365425.1 B4GALT1
[organism = Homo sapiens]
[GeneID = 2683]
[isoform = 3])
SEQ ID NO: 7
MRLREPLLSGSAAMPGASLQRACRLLVAVCAL
HLGVTLVYYLAGRDLSRLPQLVGVSTPLQGGS
NSAAAIGQSSGELRTGGARPPPPLGASSQPRP
GGDSSPVVDSGPGPASNLTSVPVPHTTALSLP
ACPEESPLLVGPMLIEFNMPVDLELVAKQNPN
VKMGGRYAPRDCVSPHKVAIIIPFRNRQEHLK
YWLYYLHPVLQRQQLDYGIYVINQAGDTIFNR
AKLLNVGFQEALKDYDYTCFVFSDVDLIPMND
HNAYRCFSQPRHISVAMDKFGFRLVFRGMSIS
RPNAVVGRCRMIRHSRDKKNEPNPQRFDRIAH
TKETMLSDGLNSLTYQVLDVQRYPLYTQITVD
IGTPS
(NP_001365426.1 B4GALT1
[organism = Homo sapiens]
[GeneID = 2683]
[isoform = 4])
SEQ ID NO: 8
MRLREPLLSGSAAMPGASLQRACRLLVAVCAL
HLGVTLVYYLAGRDLSRLPQLVGVSTPLQGGS
NSAAAIGQSSGELRTGGARPPPPLGASSQPRP
GGDSSPVVDSGPGPASNLTSVPVPHTTALSLP
ACPEESPLLVGPMLIEFNMPVDLELVAKQNPN
VKMGGRYAPRDCVSPHKVAIIIPFRNRQEHLK
YWLYYLHPVLQRQQLDYGIYVINQYEKIRRLL
W
SEQ ID NO: 9
MRLREPLLSGSAAMPGASLQRACR
SEQ ID NO: 10
LLVAVCALHLGVTLVYYLAG
SEQ ID NO: 11
RDLSRLPQLVGVSTPLQGGSNSAAAIGQSSGE
LRTGGARPPPPLGASSQPRPGGDSSPVVDSGP
GPASNLTSVPVPHTTALSLPACPEESPLLVGP
MLIEFNMPVDLELVAKQNPNVKMGGRYAPRDC
VSPHKVAIIIPFRNRQEHLKYWLYYLHPVLQR
QQLDYGIYVINQAGDTIFNRAKLLNVGFQEAL
KDYDYTCFVFSDVDLIPMNDHNAYRCFSQPRH
ISVAMDKFGFSLPYVQYFGGVSALSKQQFLTI
NGFPNNYWGWGGEDDDIFNRLVFRGMSISRPN
AVVGRCRMIRHSRDKKNEPNPQRFDRIAHTKE
TMLSDGLNSLTYQVLDVQRYPLYTQITVDIGT
PS
(B4GalT)
SEQ ID NO: 12
GPASNLTSVPVPHTTALSLPACPEESPLLVGP
MLIEFNMPVDLELVAKQNPNVKMGGRYAPRDC
VSPHKVAIIIPFRNRQEHLKYWLYYLHPVLQR
QQLDYGIYVINQAGDTIFNRAKLLNVGFQEAL
KDYDYTCFVFSDVDLIPMNDHNAYRCFSQPRH
ISVAMDKFGFSLPYVQYFGGVSALSKQQFLTI
NGFPNNYWGWGGEDDDIFNRLVFRGMSISRPN
AVVGRCRMIRHSRDKKNEPNPQRFDRIAHTKE
TMLSDGLNSLTYQVLDVQRYPLYTQITVDIGT
PS
(B4GalT)
SEQ ID NO: 13
gssplldmGPASNLTSVPVPHTTALSLPACPE
ESPLLVGPMLIEFNMPVDLELVAKQNPNVKMG
GRYAPRDCVSPHKVAIIIPFRNRQEHLKYWLY
YLHPVLQRQQLDYGIYVINQAGDTIFNRAKLL
NVGFQEALKDYDYTCFVFSDVDLIPMNDHNAY
RCFSQPRHISVAMDKFGFSLPYVQYFGGVSAL
SKQQFLTINGFPNNYWGWGGEDDDIFNRLVFR
GMSISRPNAVVGRCRMIRHSRDKKNEPNPQRF
DRIAHTKETMLSDGLNSLTYQVLDVQRYPLYT
QITVDIGTPSprdhhhhhhh
(NP_001340845.1
(NP_003023.1, NP_775323.1) ST6GAL1
[organism = Homo sapiens]
[GeneID = 6480]
[isoform = a])
SEQ ID NO: 14
MIHTNLKKKFSCCVLVFLLFAVICVWKEKKKG
SYYDSFKLQTKEFQVLKSLGKLAMGSDSQSVS
SSSTQDPHRGRQTLGSLRGLAKAKPEASFQVW
NKDSSSKNLIPRLQKIWKNYLSMNKYKVSYKG
PGPGIKFSAEALRCHLRDHVNVSMVEVTDFPF
NTSEWEGYLPKESIRTKAGPWGRCAVVSSAGS
LKSSQLGREIDDHDAVLRFNGAPTANFQQDVG
TKTTIRLMNSQLVTTEKRFLKDSLYNEGILIV
WDPSVYHSDIPKWYQNPDYNFFNNYKTYRKLH
PNQPFYILKPQMPWELWDILQEISPEEIQPNP
PSSGMLGIIIMMTLCDQVDIYEFLPSKRKTDV
CYYYQKFFDSACTMGAYHPLLYEKNLVKHLNQ
GTDEDIYLLGKATLPGFRTIHC
(NP_775324.1 ST6GAL1
[organism = Homo sapiens]
[GeneID = 6480]
[isoform = b])
SEQ ID NO: 15
MNSQLVTTEKRFLKDSLYNEGILIVWDPSVYH
SDIPKWYQNPDYNFFNNYKTYRKLHPNQPFYI
LKPQMPWELWDILQEISPEEIQPNPPSSGMLG
IIIMMTLCDQVDIYEFLPSKRKTDVCYYYQKF
FDSACTMGAYHPLLYEKNLVKHLNQGTDEDIY
LLGKATLPGFRTIHC
SEQ ID NO: 16
MIHTNLKKK
SEQ ID NO: 17
FSCCVLVFLLFAVICVW
SEQ ID NO: 18
KEKKKGSYYDSFKLQTKEFQVLKSLGKLAMGS
DSQSVSSSSTQDPHRGRQTLGSLRGLAKAKPE
ASFQVWNKDSSSKNLIPRLQKIWKNYLSMNKY
KVSYKGPGPGIKFSAEALRCHLRDHVNVSMVE
VTDFPFNTSEWEGYLPKESIRTKAGPWGRCAV
VSSAGSLKSSQLGREIDDHDAVLRFNGAPTAN
FQQDVGTKTTIRLMNSQLVTTEKRFLKDSLYN
EGILIVWDPSVYHSDIPKWYQNPDYNFFNNYK
TYRKLHPNQPFYILKPQMPWELWDILQEISPE
EIQPNPPSSGMLGIIIMMTLCDQVDIYEFLPS
KRKTDVCYYYQKFFDSACTMGAYHPLLYEKNL
VKHLNQGTDEDIYLLGKATLPGFRTIHC
(ST6Gal1)
SEQ ID NO: 19
AKPEASFQVWNKDSSSKNLIPRLQKIWKNYLS
MNKYKVSYKGPGPGIKFSAEALRCHLRDHVNV
SMVEVTDFPFNTSEWEGYLPKESIRTKAGPWG
RCAVVSSAGSLKSSQLGREIDDHDAVLRFNGA
PTANFQQDVGTKTTIRLMNSQLVTTEKRFLKD
SLYNEGILIVWDPSVYHSDIPKWYQNPDYNFF
NNYKTYRKLHPNQPFYILKPQMPWELWDILQE
ISPEEIQPNPPSSGMLGIIIMMTLCDQVDIYE
FLPSKRKTDVCYYYQKFFDSACTMGAYHPLLY
EKNLVKHLNQGTDEDIYLLGKATLPGFRTIHC
(ST6Gal1)
SEQ ID NO: 20
gssplldmlehhhhhhhhmAKPEASFQVWNKD
SSSKNLIPRLQKIWKNYLSMNKYKVSYKGPGP
GIKFSAEALRCHLRDHVNVSMVEVTDFPFNTS
EWEGYLPKESIRTKAGPWGRCAVVSSAGSLKS
SQLGREIDDHDAVLRFNGAPTANFQQDVGTKT
TIRLMNSQLVTTEKRFLKDSLYNEGILIVWDP
SVYHSDIPKWYQNPDYNFFNNYKTYRKLHPNQ
PFYILKPQMPWELWDILQEISPEEIQPNPPSS
GMLGIIIMMTLCDQVDIYEFLPSKRKTDVCYY
YQKFFDSACTMGAYHPLLYEKNLVKHLNQGTD
EDIYLLGKATLPGFRTIHC

1. A method of producing hypersialylated IgG (hsIgG), the method comprising:
(a) providing pooled IgG antibodies;
(b) incubating the pooled IgG antibodies in a reaction mixture comprising β1,4-Galactosyltransferase (B4GalT) or enzymatically active portion thereof, UDP-Gal or salt thereof, Bis (2-hydroxyethyl) aminotris (hydroxymethyl)methane (BIS-TRIS) buffer, and MnCl₂, thereby producing galactosylated IgG antibodies; and
(c) incubating the galactosylated IgG antibodies in a reaction mixture comprising ST6Gal or enzymatically active portion thereof, CMP-NANA or salt thereof, Bis (2-hydroxyethyl) aminotris (hydroxymethyl)methane (BIS-TRIS) buffer, and MnCl₂,
thereby producing hsIgG.

2. A method of preparing hypersialylated IgG (hsIgG), the method comprising:
(a) providing pooled IgG antibodies;
(b) incubating the pooled IgG antibodies in a reaction mixture comprising β1,4-Galactosyltransferase (B4GalT) or enzymatically active portion thereof, UDP-Gal or salt thereof, ST6Gal or enzymatically active portion thereof, CMP-NANA or salt thereof, Bis (2-hydroxyethyl) aminotris (hydroxymethyl)methane (BIS-TRIS) buffer, and MnCl₂, thereby creating the hsIgG preparation.

3. A method of preparing hypersialylated IgG (hsIgG), the method comprising:
(a) providing pooled IgG antibodies;
(b) incubating the pooled IgG antibodies in a galactosylation reaction mixture comprising β1,4-Galactosyltransferase (B4GalT) or enzymatically active portion thereof, UDP-Gal or salt thereof, Bis (2-hydroxyethyl) aminotris (hydroxymethyl)methane (BIS-TRIS) buffer, and MnCl₂, thereby producing galactosylated IgG antibodies;
(c) adding ST6Gal or an enzymatically active portion thereof and CMP-NANA or salt thereof to the galactosylation reaction mixture to produce a sialylation reaction mixture; and
(d) incubating the sialylation reaction mixture,
thereby producing hsIgG

4. The method of claim 1, wherein the B4GalT or enzymatically active portion thereof is identical to SEQ ID NO: 13.

5. The method of claim 1, wherein the ST6Gal1 or enzymatically active portion thereof comprises an amino acid sequence that is identical to SEQ ID NO: 19.

6. The method of claim 1, wherein the total incubation time is less than 72 hours.

7. (canceled)

8. The method of claim 1, wherein each of the reaction mixture(s) each independently comprise BIS-TRIS at from about 10 to about 500 mM and from about pH 5.5 to about pH 8.5.

9. (canceled)

10. (canceled)

11. The method of claim 1 s, wherein each of the reaction mixture(s) each independently comprise MnCl₂ at about 1 to about 20 mM.

12. The method of claim 1, wherein each of the reaction mixture(s) each independently comprise MnCl₂ at about 4.5 to about 5.5 mM.

13. The method of claim 1, wherein the reaction mixture comprises from about 0.038 to about 0.046 UDP-Gal or salt thereof per gram of pooled IgG antibodies.

14. The method of claim 1, wherein the reaction mixture comprises about 0.1425 to about 0.1575 CMP-NANA or salt thereof per gram of IgG antibody.

15. The method of claim 1, wherein the reaction mixture comprising CMP-NANA is supplemented with additional CMP-NANA or salt thereof during incubation.

16. The method of claim 15, wherein the total amount of CMP-NANA or salt thereof added to the reaction mixture comprising CMP-NANA is from about 0.1425 to about 0.1575.

17. (canceled)

18. The method of claim 1, wherein the reaction mixture comprising B4GalT or enzymatically active portion thereof comprises from about 7.2 to or to about 8.8 U B4GalT or enzymatically active portion thereof per gram of pooled IgG.

19. The method of claim 1, wherein the reaction mixture comprising ST6Gal or enzymatically active portion thereof comprises from about 17.1 to about 18.9 U ST6Gal1 or enzymatically active portion thereof per gram of pooled IgG.

20. The method of claim 1, wherein the incubation takes place at from about 20 to about 50° C.

21. (canceled)

22. The method of claim 1, wherein the IgG antibodies comprise IgG antibodies isolated from at least 1000 donors.

23. The method of claim 1, wherein at least 50%, 55%, 60%, 65% or 70% w/w of the IgG antibodies are IgG1 antibodies.

24. (canceled)

25. (canceled)

26. (canceled)

27. The method of claim 1, wherein at least 60%, 65%, 70%, 75%, 80%, or 85% of the branched glycans on the Fab domain of the hsIgG have a sialic acid on both the α1,3 arm and the α 1,6 arm that is connected through a NeuAc-α 2,6-Gal terminal linkage.

28. (canceled)

29. (canceled)

30. (canceled)

31. (canceled)

32. The method of claim 1, wherein at least 90% of the branched Fc glycans on the hsIgG have a sialic acid on both the α1,3 branch and the α1,6 branch.

33. (canceled)
